(19) United States
(12) Patent Application Publication
(10) Pub. No.: US 2015/0190499 A1
(43) Pub. Date: Jul. 9, 2015

(54) BOVINE INFLUENZA C VIRUS COMPOSITIONS

(71) Applicant: Zoetis LLC, Florham Park, NJ (US)

(72) Inventor: Siao-Kun W. Welch, Kalamazoo, MI (US)

(73) Assignee: Zoetis LLC, Florham Park, NJ (US)

(21) Appl. No.: 14/415,328

(22) PCT Filed: Jul. 18, 2013
(86) PCT No.: PCT/US2013/050982

§ 371 Date: Jan. 16, 2015

Related U.S. Application Data
(60) Provisional application No. 61/729,736, filed on Nov. 26, 2012.
(60) Provisional application No. 61/673,298, filed on Jul. 19, 2012.

(51) Int. Cl. A61K 39/145; C12N 7/00; G01N 33/569; C07K 16/10

(52) U.S. Cl. CPC A61K 39/145; C07K 16/1018; C12N 7/00; G01N 33/56983; C12N 2760/16334; G01N 2333/11; G01N 2469/10

(57) ABSTRACT

An influenza C virus has been isolated from a bovine species. Influenza C virus polynucleotides and polypeptides have also been identified. Immunogenic compositions are also described, as well as diagnostic kits and methods of detection.

### FIELD OF THE INVENTION

The present application relates to the field of microbiology and immunology, in particular to a virus and immunogenic compositions comprising it. Specifically, it relates to an influenza C virus isolated from a bovine. Also disclosed herein are influenza C virus polynucleotides and polypeptides. Finally, diagnostic kits and methods of detection are disclosed.

### BACKGROUND

Bovine respiratory disease (BRD) complex is the most significant health problem of the beef industry. In 1991, an estimated loss of $624 million occurred, due to costs of treatment, production loss, and death. BRD complex is a multifactorial infection, having many contributing pathogens, both viral and bacterial.

Virus families containing enveloped single-stranded RNA of the negative-sense genome are classified into groups having non-segmented genomes (Paramyxoviridae, Rhabdoviridae) or those having segmented genomes (orthomyxoviridae, Bunyaviridae and Arenaviridae). The Orthomyxoviridae family contains only the viruses of influenza, types A, B and C.

The influenza virions consist of an internal ribonucleoprotein core (a helical nucleocapsid) containing the segmented, single-stranded RNA genome, and an outer lipoprotein envelope lined inside by a matrix protein (M). The segmented genome of influenza A consists of eight molecules (seven for influenza C) of linear, negative polarity, single-stranded RNAs which encode ten polypeptides, including: the RNA-directed RNA polymerase proteins (PB2, PB1 and PA) and nucleoprotein (NP) which form the nucleocapsid; the matrix proteins (M1, M2); two surface glycoproteins which project from the lipoprotein envelope: hemagglutinin (HA) and neuraminidase (NA) which is lacking for the influenza C virus; and nonstructural proteins whose function is unknown (NS1 and NS2). Transcription and replication of the genome takes place in the nucleus and assembly occurs via budding on the plasma membrane. The viruses can reassort genes during mixed infections.

Influenza C virus has only one species in this genus, referred to as “influenza C virus”. Influenza C viruses have been isolated from humans and pigs. Influenza A and B viruses are the causative agents of an infection commonly referred to as the “flu”, which can produce clinical symptoms of chills, fever, sore throat, muscle pains, headache, coughing, weakness/fatigue and general discomfort. Influenza C virus infection, however, is rare compared to type A or B, and causes mild upper respiratory infection in human or the infection is unapparent.

### SUMMARY OF THE INVENTION

The applicants have surprisingly identified, and disclose herein, an influenza C virus isolated from a bovine. Disclosed and provided herein are polynucleotides and polypeptides of said bovine influenza C virus, as well as immunogenic compositions comprising said polynucleotides and or said polypeptides. Diagnostic kits and methods of detecting a bovine influenza C virus are also provided.

In one embodiment, the disclosure provides an isolated bovine influenza C virus.

In another aspect, the invention provides a composition comprising an isolated influenza C virus, wherein said virus comprises at least one of the following gene segments a nucleic acid encoding a protein having greater than 52% identity to SEQ ID NO: 4; a nucleic acid encoding a protein having greater than 72% identity to SEQ ID NO: 6; a nucleic acid encoding a protein having greater than 50% identity to SEQ ID NO: 8; a nucleic acid encoding a protein having greater than 54% identity to SEQ ID NO: 10; a nucleic acid encoding a protein having greater than 40% identity to SEQ ID NO: 12; a nucleic acid encoding a protein having greater than 36% identity to SEQ ID NO: 14; and a nucleic acid encoding a protein having greater than 33% identity to SEQ ID NO: 16.

In another aspect, the instant invention provides a composition comprising an isolated nucleic acid comprising one or more of the nucleic acids: a nucleic acid encoding a protein having greater than 52% identity to SEQ ID NO: 4; a nucleic acid encoding a protein having greater than 72% identity to SEQ ID NO: 6; a nucleic acid encoding a protein having greater than 50% identity to SEQ ID NO: 8; a nucleic acid encoding a protein having greater than 54% identity to SEQ ID NO: 10; a nucleic acid encoding a protein having greater than 40% identity to SEQ ID NO: 12; a nucleic acid encoding a protein having greater than 36% identity to SEQ ID NO: 14; and a nucleic acid encoding a protein having greater than 33% identity to SEQ ID NO: 16, wherein SEQ ID NO:4 encodes an influenza C “PB2” protein; SEQ ID NO:6 encodes an influenza C “PB1” protein; SEQ ID NO: 8 encodes an influenza “PA” protein; SEQ ID NO:10 encodes an influenza C “HE” protein; SEQ ID NO:12 encodes an influenza C “N” protein; SEQ ID NO:14 encodes an influenza C “M” protein; and SEQ ID NO:16 encodes an influenza C “NS1” protein.

In additional aspects, the invention provides immunogenic compositions, expression vectors and host cells comprising bovine influenza C virus, and/or amino acids and/or nucleic acids according to the previously mentioned aspects of the invention.

In yet another aspect, the invention provides a method of treating or protecting an animal from disease caused by an influenza C virus, the method comprising administering to the animal the immunogenic composition comprising the virus, or the nucleic acid sequence(s) or the amino acid sequence(s) according to the aspects of the invention disclosed above.

In other aspects, the invention provides diagnostic kits and methods, and antibodies and/or nucleotide primers useful for such methods and kits. More specifically, in some aspects, the invention provides a method of detecting exposure of an animal to influenza C virus that comprises determining the presence of any one or more of SEQ ID NO:'s 3; 5; 7; 9; 11; 13; and 15 in a sample from the animal.

In another aspect, the invention provides a method of detecting exposure of an animal to influenza C virus that comprises determining the presence of any one or more of SEQ ID NO:'s 4; 6; 8; 10; 12; 14; and 16 in a sample from the animal.

In yet another aspect, the invention provides n antibody that specifically binds to the isolated bovine influenza C virus.

In another aspect, the invention also provides an antibody that specifically binds to an epitope from a polypeptide selected from the polypeptides of SEQ ID NO:'s 4; 6; 8; 10; 12; 14; and 16-20.

### BRIEF DESCRIPTION OF THE FIGURES

FIG. 1 is an electron micrograph of the isolated bovine influenza C virus.

### DETAILED DESCRIPTION

All publications, patent applications, patents, and other references mentioned herein are incorporated by reference in their entirety.

The following definitions may be applied to terms employed in the description of the embodiments. The following definitions supersede any contradictory definitions contained in each individual reference incorporated herein by reference.

Unless otherwise defined herein, scientific and technical terms used in connection with the present embodiments shall have the meanings that are commonly understood by those of ordinary skill in the art. Further, unless otherwise required by context, singular terms shall include pluralities, and plural terms shall include the singular.

The terms “about” or “approximately”, as used herein, when used in connection with a measurable numerical variable, mean the indicated value of the variable, and all values of the variable that are within the experimental error of the indicated value (e.g., within the 95% confidence interval for the mean), or within 10 percent of the indicated value, whichever is greater.

The term “adjuvant”, as used herein, means a pharmacological or immunological agent that modifies the effect of other agents, such as a drug or immunogenic composition. Adjuvants are often included in immunogenic compositions to enhance the recipient's immune response to a supplied antigen. See below for a further description of adjuvants. The term “amino acid”, as used herein, refers to naturally-occurring and synthetic amino acids, as well as amino acid analogs and amino acid mimetics that function in a manner similar to naturally-occurring amino acids. Naturally-occurring amino acids are those encoded by the genetic code, as well as those amino acids that are later modified, for example, hydroxyproline, carboxyglutamate, and O-phosphoserine. Stereoisomers (e.g., D-amino acids) of the twenty conventional amino acids, unnatural amino acids such as α and α-disubstituted amino acids, N-alkyl amino acids, and other unconventional amino acids, may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids.

Amino acid analogs refer to compounds that have the same basic chemical structure as a naturally-occurring amino acid, i.e., a carbon that is bound to a hydrogen, a carboxyl group, an amino group, and an R group. Exemplary amino acid analogs include, for example, homoserine, norleucine, methionine sulfoxide, and methionine methyl sulfonium. Such analogs have modified R groups (e.g., norleucine) or modified peptide backbones, but retain the same essential chemical structure as a naturally-occurring amino acid. Amino acid mimetics refer to chemical compounds that have a structure that is different from the general chemical structure of an amino acid, but that function in a manner similar to a naturally-occurring amino acid.

Amino acids may be referred to herein by either their commonly known three-letter symbols or their one-letter symbols recommended by the IUPAC-IUB Biochemical Nomenclature Commission.

The term “conservative amino acid substitution” as used herein, refers to any amino acid substitution for a given amino acid residue, where the substitute residue is so chemically similar to that of the given residue that no substantial decrease in polypeptide function (e.g., enzymatic activity) results. Conservative amino acid substitutions are commonly known in the art, and examples thereof are described, e.g., in U.S. Pat. Nos. 6,790,639, 6,774,107, 6,194,167, or 5,350,576. In a preferred embodiment, a conservative amino acid substitution will be anyone that occurs within one of the following six groups:

Small aliphatic, substantially non-polar residues: Ala, Gly, Pro, Ser, and Thr;

Large aliphatic, non-polar residues: Ile, Leu, Val, and Met;

Polar, negatively-charged residues: Asp and Glu;

Amides of polar, negatively-charged residues: Asn and Gln;

Polar, positively-charged residues: Arg, Lys, and His; and

Large aromatic residues: Trp, Tyr, and Phe.

In a preferred embodiment, a conservative amino acid substitution will be any one of the following, which are listed as Native Residue (Conservative Substitutions) pairs: Ala (Ser); Arg (Lys); Asn (Gln; His); Asp (Glu); Gln (Asn); Glu (Asp); Gly (Pro); His (Asn; Gln); Ile (Leu; Val); Leu (Ile; Val); Lys (Arg; Gln; Glu); Met (Leu; Ile); Phe (Met; Leu; Tyr); Ser (Thr); Thr (Ser); Trp (Tyr); Tyr (Trp; Phe); and Val (Ile; Leu).

The term “animal”, as used herein, means any animal that is susceptible to infection by bovine influenza C virus, including mammals, both domesticated and wild. Preferably, “animal”, as used herein, refers to a bovine.

The terms “antibody” or “antibodies”, as used herein, mean an immunoglobulin molecule able to bind to an antigen by means of recognition of an epitope. Immunoglobulins are serum proteins composed of “light” and “heavy” polypeptide chains, which have “constant” and “variable” regions, and are divided into classes (e.g., IgA, IgD, IgE, IgG, and IgM) based on the composition of the constant regions. An antibody that is “specific” for a given antigen indicates that the variable regions of the antibody recognize and bind a particular antigen exclusively. Antibodies can be a polyclonal mixture, or monoclonal. They can be intact immunoglobulins derived from natural or recombinant sources, or can be immunoreactive portions of intact immunoglobulins. Antibodies can exist in a variety of forms, including Fv, Fab′, F(ab′)2, Fc, as well as single chain. An antibody can be converted to an antigen-binding protein, which includes, but is not limited to, antibody fragments. As used herein, the term “antigen binding protein”, “antibody” and the like, which may be used interchangeably, refer to a polypeptide or polypeptides, or fragment(s) thereof, comprising an antigen binding site. The term “antigen binding protein” or “antibody” preferably refers to monoclonal antibodies and fragments thereof, and immunologic-binding equivalents thereof that can bind to a particular protein and fragments thereof. As used herein, the term encompasses not only intact polyclonal or monoclonal antibodies, but also fragments thereof. For the purposes of the present invention, “antibody” and “antigen binding protein” also includes antibody fragments, unless otherwise stated. Exemplary antibody fragments include Fab, Fab′, F(ab′)2, Fv, scFv, Fd, dAb, diabodies, their antigen-recognizing fragments, small modular immunopharmaceuticals (SMIPs) nanobodies, IgNAR (immunoglobulin new antigen receptor) molecules and the like, all recognized by one of skill in the art to be an antigen binding protein or antibody fragment, and any of above-mentioned fragments and their chemically or genetically manipulated counterparts, as well as other antibody fragments and mutants thereof, fusion proteins comprising an antibody portion, and any other modified configuration of the immunoglobulin molecule that comprises an antigen recognition site. Antibodies and antigen binding proteins can be made, for example, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)). For various other antibody production techniques, see Antibodies: A Laboratory Manual, eds. Harlow et al., Cold Spring Harbor Laboratory, 1988 as well as other techniques that are well known to those skilled in the art.

The term “specifically binds,” “binds specifically” or “specific binding”, in the context of antibody binding, refers to high avidity and/or high affinity binding of an antibody to a specific antigen, i.e., a polypeptide, or epitope. Antibody specifically binding an antigen is stronger than binding of the same antibody to other antigens. Antibodies which bind specifically to a polypeptide may be capable of binding other polypeptides at a weak, yet detectable level (e.g., 10% or less of the binding shown to the polypeptide of interest). Such weak binding, or background binding, is readily discernable from the specific antibody binding to a subject polypeptide, e.g. by use of appropriate controls. In general, specific antibodies bind to an antigen with a binding affinity with a K_{d} of 10⁻⁷ M or less, e.g., 10⁻⁸M or less e.g., 10⁻⁹ M or less, 10⁻¹⁰ or less, 10⁻¹¹ or less, 10⁻¹² or less, or 10⁻¹³ or less, etc.

“Antigen”, as used herein, means a molecule that contains one or more epitopes (linear, conformational or both), that upon exposure to a subject, will induce an immune response that is specific for that antigen. An epitope is the specific site of the antigen which binds to a T-cell receptor or specific B-cell antibody, and typically comprises about 3 to about 20 amino acid residues. The term “antigen” can also refer to subunit antigens—antigens separate and discrete from a whole organism with which the antigen is associated in nature—as well as killed, attenuated or inactivated bacteria, viruses, fungi, parasites or other microbes. The term “antigen” also refers to antibodies, such as anti-idiotype antibodies or fragments thereof, and to synthetic peptide mimotopes that can mimic an antigen or antigenic determinant (epitope). The term “antigen” also refers to an oligonucleotide or polynucleotide that expresses an antigen or antigenic determinant in vivo, such as in DNA immunization applications. An “antigen”, as used herein, is a molecule or a portion of a molecule capable of being specifically bound by an antibody or antigen binding protein. In particular, an antibody, or antigen binding protein, will bind to epitopes of the antigen. An epitope, as used herein, refers to the antigenic determinant recognized by the hypervariable region, or Complentarity Determining Region (CDR), of the variable region of an antibody or antigen binding protein. Unless indicated otherwise, the term “epitope” as used herein, refers to a region of the Bovine Influenza Virus C that will specifically bind to an antibody of the invention.

The term “bovine”, as used herein, means a diverse group of medium—to large-sized ungulates, generally having cloven hoofs, and at least one of the sexes having true horns. Bovines include, but are not limited to, domestic cattle, bison, African buffalo, water buffalo, yak, and four-horned or spiral-horned antelope.

The terms “diagnose”, “diagnosing” or “diagnostic”, as used herein, mean the identification of the nature and/or cause of something, such as a disease, or a kit which is useful for making such identification.

The term “gene segment”, as used herein, means a piece of nucleic acid which is part of a virus genome. In the case of influenza viruses, they contain seven or eight gene segments, some of which contain more than one gene. A viral genome composed of gene segments is either all DNA or all RNA.

The term “heterologous”, as used herein, means a combination of elements not naturally occurring. For example, heterologous DNA refers to DNA not naturally located in the cell, or at a chromosomal site in the cell. Heterologous DNA can also include a gene foreign to the cell. A “heterologous expression regulatory element,” or “heterologous promoter”, is an element operably associated with a different gene than the one it is associated with in nature. As used herein, a “heterologous nucleotide sequence” refers to a nucleotide sequence that is added to a nucleotide sequence of the present invention by recombinant methods to form a nucleic acid which is not naturally formed in nature. Such nucleic acids can encode chimeric and/or fusion proteins/polypeptides. Thus the heterologous nucleotide sequence can encode peptides/proteins that contain regulatory and/or structural properties.

The term “host cell”, as used herein, means a prokaryotic or eukaryotic cell that harbors a plasmid, vector, or virus. Such cells may include, but are not limited to, bacterial cells, yeast cells, insect cells, animal cells, and mammalian cells (e.g., murine, rat, simian, or human). The term “host cell” can mean any individual cell or cell culture capable of supporting replication of a virus. With respect to plasmids and vectors, a “host cell” is any individual cell or cell culture which can be or has been a recipient for vectors, or for the incorporation of exogenous nucleic acid molecules, polynucleotides, and/or proteins. It also is intended to include progeny of a single cell. The progeny may not necessarily be completely identical in morphology, or in genomic or total DNA complement, to the original parent cell due to natural, accidental, or deliberate mutation. A “host cell” is intended to include any individual cell or cell culture that can be or has been a recipient for vectors or for the incorporation of exogenous nucleic acid molecules, polynucleotides, and/or proteins. It also is intended to include progeny of a single cell. The progeny may not necessarily be completely identical (in morphology or in genomic or total DNA complement) to the original parent cell due to natural, accidental, or deliberate mutation. As used herein, the terms “host cell”, “cell”, “cell line”, and “cell culture” may be used interchangeably.

The term “identity”, as used herein, means the extent to which two nucleotide or protein sequences are invariant. The term “similarity” or “homology”, as used herein, means the extent to which nucleotide or protein sequences are related. The extent of similarity between two sequences can be based on percent sequence identity and/or conservation. Amino acids other than those indicated as conserved may differ in a protein or enzyme, so that the percent protein or amino acid sequence similarity between any two proteins of similar function may vary and can be, for example, at least 70%, 75%, 80%, 85%, 90%, and 95%, as determined according to an alignment scheme.

The term “immunogenic composition”, as used herein, means a composition that generates an immune response (i.e., has immunogenic activity) when administered alone, or with a pharmaceutically-acceptable carrier, to an animal. The immune response can be a cellular immune response mediated primarily by cytotoxic T-cells, or a humoral immune response mediated primarily by helper T-cells, which in turn activate B-cells, leading to antibody production. In addition, specific T-lymphocytes or antibodies can be generated to allow for the future protection of an immunized host.

The terms “influenza C virus”, “type C influenzavirus”, or “influenzavirus C”, as used herein, mean a genus within the virus family Orthomyxoviridae, which includes those viruses which can cause influenza, commonly referred to as the “flu”.

The term “isolated”, as used herein, means that the referenced material is removed from the environment in which it is normally found. Thus, an isolated biological material can be free of cellular components, i.e., components of the cells in which the material is found or produced. In the case of nucleic acid molecules, an isolated nucleic acid includes, for example, a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes located upstream or downstream of the nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified. An “isolated” or “purified” polypeptide or polynucleotide, e.g., an “isolated polypeptide,” or an “isolated polynucleotide”, is purified to a state beyond that in which it exists in nature. For example, the “isolated” or “purified” polypeptide or polynucleotide can be substantially free of cellular material or other contaminating proteins from the cell or tissue source from which the protein or polynucleotide is derived, or substantially free from chemical precursors or other chemicals when chemically synthesized. The preparation of antigen binding protein having less than about 50% of non-antigen binding protein (also referred to herein as a “contaminating protein”), or of chemical precursors, is considered to be “substantially free.” 40%, 30%, 20%, 10% and more preferably 5% (by dry weight), of non-antigen binding protein, or of chemical precursors, is considered to be substantially free.

The term “operably linked”, as used herein, means that a nucleic acid molecule, e.g., DNA or RNA, and one or more regulatory expression elements (e.g., a promoter or portion thereof with or without an enhancer, an Internal ribosome entry site (IRES) or other expression regulatory element are connected in such a way as to permit transcription of an RNA from the nucleic acid molecule, or permit expression of the product (i.e., a polypeptide) of the nucleic acid molecule, when the appropriate molecules are bound to the regulatory sequences. Regulatory expression elements can be configured to generate one or more double-strand or single-strand nucleic acid(s), in plus or minus orientation.

The terms “peptide”, “polypeptide”, or “protein”, as used herein, mean an organic polymer molecule composed of two or more amino acids bonded in a chain. The terms “polypeptide”, “peptide”, and “protein”, are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The term “plasmid”, as used herein, means a genetic element that is stably inherited without being a part of the chromosome of its host cell. Plasmids may be comprised of DNA or RNA, and may be linear or circular. Plasmids code for molecules that ensure their replication and stable inheritance during cell replication, and may encode products of medical, agricultural and environmental importance. Plasmids are widely used in molecular biology as vectors to clone and express recombinant genes.

The terms “polynucleotide” or “polynucleotide molecule”, as used herein, mean an organic polymer molecule composed of nucleotide monomers covalently bonded in a chain. DNA (deoxyribonucleic acid) and RNA (ribonucleic acid) are examples of polynucleotides with distinct biological function. The terms “nucleic acid”, “polynucleotide”, “nucleic acid molecule” and the like may be used interchangeably herein, and refer to a series of nucleotide bases (also called “nucleotides”) in DNA and RNA. The nucleic acid may contain deoxyribonucleotides, ribonucleotides, and/or their analogs. The term “nucleic acid” includes, for example, single-stranded and double-stranded molecules. A nucleic acid can be, for example, a gene or gene fragment, exons, introns, a DNA molecule (e.g., cDNA), an RNA molecule (e.g., mRNA), recombinant nucleic acids, plasmids, and other vectors, primers and probes. Both 5′ to 3′ (sense) and 3′ to 5′ (antisense), polynucleotides are included. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps” (substitution of one or more of the naturally occurring nucleotides with an analog), internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl, 2′-fluoro- or 2′-azido-ribose, carbocyclic sugar analogs, anomeric sugars, epimeric sugars such as arabinose, xylose or lyxose, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C), optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “prevent”, “preventing” or “prevention”, and the like, as used herein, mean to inhibit the replication of a microorganism, to inhibit transmission of a microorganism, or to inhibit a microorganism from establishing itself in its host. These terms, and the like, can also mean to inhibit or block one or more signs or symptoms of infection.

The terms “recombinant protein” or “recombinant”, as used herein, mean proteins, peptides or polypeptides derived, and the techniques used to produce them, from cells transformed by an exogenous DNA construct encoding the desired protein, peptide or polypeptide.

The term “therapeutically effective amount” (or “effective amount”) refers to an amount of an active ingredient, e.g., an agent according to the invention, sufficient to effect beneficial or desired results when administered to a subject or patient. An effective amount can be administered in one or more administrations, applications or dosages. A therapeutically effective amount of a composition according to the invention may be readily determined by one of ordinary skill in the art.

As used herein, the terms “therapeutic” or “treatment” encompass the full spectrum of treatments for a disease or disorder. By way of example, a “therapeutic” agent of the invention may act in a manner, or a treatment may result in an effect, that is prophylactic or preventive, including those that incorporate procedures designed to target animals that can be identified as being at risk (pharmacogenetics); or in a manner that is ameliorative or curative in nature; or may act to slow the rate or extent of the progression of at least one symptom of a disease or disorder being treated.

The term “vector”, as used herein, refers to a polynucleotide molecule capable of carrying and transferring another polynucleotide fragment or sequence to which it has been linked from one location (e.g., a host, a system) to another. The term includes vectors for in vivo or in vitro expression systems. For example, vectors of the invention can be in the form of “plasmids”, which refer to circular double-stranded DNA loops which are typically maintained episomally, but may also be integrated into the host genome. Vectors of the invention can also be in linear forms. In addition, the invention is intended to include other forms of vectors which serve equivalent functions, and which become known in the art subsequently hereto.

The term “veterinarily-acceptable carrier”, as used herein, refers to substances which are, within the scope of sound medical judgment, suitable for use in contact with the tissues of animals, without undue toxicity, irritation, allergic response, and the like, commensurate with a reasonable benefit-to-risk ratio, and effective for their intended use.

The following description is provided to aid those skilled in the art in practicing the present invention. Even so, this description should not be construed to unduly limit the present invention, as modifications and variations in the embodiments discussed herein can be made by those of ordinary skill in the art, without departing from the spirit or scope of the present inventive discovery.

### Viruses; Immunogenic Compositions

In certain embodiments of the present invention, an influenza C virus has been identified and isolated from a bovine, as well as characterized. An isolate of influenza C virus was deposited with the ATCC, and has been assigned accession number, PTA-13105.

In one aspect, the influenza C virus of the invention is characterized as comprising at least one of the following nucleic acid sequences: a nucleic acid sequence encoding PB2 protein, PB1 protein, PA protein, HE protein, N protein, M protein, and NS1 protein. Any combination of these seven is also encompassed by the instant invention.

The HE protein of the instant invention comprises SEQ ID NO: 20, or an amino acid sequence that is at least 93.2% identical, or at least 95.5% identical, or at least 98% identical to SEQ ID NO: 20.

In some embodiments, the HE protein of the instant invention comprises SEQ ID NO: 18, or a sequence that is at least 78% identical and 84% similar. In some embodiments, the identity to SEQ ID NO: 18 is at least 80%, or at least 85%, or at least 90%, or at least 95%, or at least 99%, and the similarity to SEQ ID NO: 18 is at least 85%, or at least 90%, or at least 95%, or at least 99%, optionally, with a proviso that amino acids 36-79 of SEQ ID NO: 18 are at least 93.2% identical, or at least 95.5% identical, or at least 98%, or 100% identical to the SEQ ID NO: 20.

In yet other embodiments HE protein of the instant invention comprises SEQ ID NO: 19 or a sequence that is at least 63% identical and 85% similar. In some embodiments, the identity to SEQ ID NO: 19 is at least 65%, or at least 70%, or at least 75%, or at least 80%, or at least 85%, or at least 90%, or at least 95%, or at least 99%, and the similarity to SEQ ID NO: 19 is at least 90%, or at least 95%, or at least 99%.

In other embodiments, the HE protein of the instant invention comprises an amino acid sequence that is at least 54% identical to SEQ ID NO: 10, preferably, with a proviso that amino acids 58-136 of SEQ ID NO: 10 comprise

SEQ ID NO: 18, or a sequence that is at least 78% identical and 84% similar. In some embodiments, the identity to SEQ ID NO: 18 is at least 80%, or at least 85%, or at least 90%, or at least 95%, or at least 99%, and the similarity to SEQ ID NO: 18 is at least 85%, or at least 90%, or at least 95%, or at least 99%, optionally, with a proviso that amino acids 36-79 of SEQ ID NO: 18 are at least 93.2% identical, or at least 95.5% identical, or at least 98%, or 100% identical to the SEQ ID NO: 20.

The HE protein may be further characterized by an additional or an alternative proviso that its amino acids 539-598 comprise SEQ ID NO: 19 or a sequence that is at least 63% identical and 85% similar. In some embodiments, the identity percentage to SEQ ID NO: 19 is at least 65%, or at least 70%, or at least 75%, or at least 80%, or at least 85%, or at least 90%, or at least 95%, or at least 99%, and the similarity to SEQ ID NO: 19 is at least 90%, or at least 95%, or at least 99%.

Preferably, the amino acid sequence encoding HE protein comprises a sequence that is at least 60% identical to SEQ ID NO: 10, or at least 65%, or at least 70%, or at least 75%, or at least 80%, or at least 85%, or at least 90%, or at least 95%, or at least 99%, or 100% identical to SEQ ID NO: 10.

In another aspect, the M protein is characterized as comprising SEQ ID NO: 17, or a sequence which is 67% identical and 92% similar. In some embodiments, the identity to SEQ ID NO: 17 is at least 70%, or at least 75%, at least 80%, or at least 85%, or at least 90%, or at least 95%, or at least 99%, and the similarity to SEQ ID NO: 17 is at least 95%, or at least 99%. In addition, the M protein of the influenza C virus may be characterizing as comprising an amino acid sequence that is at least 36% identical to SEQ ID NO: 14. In different embodiments, the identity may be at least 40% or at least 45%, or at least 50% or at least 55%, at least 60% or at least 65%, at least 70% or at least 75%, at least 80% or at least 85%, at least 90% or at least 95%, at least 99% or 100% identity to SEQ ID NO: 14.

The amino acid encoding the PB2 protein is characterized as having at least 52% identity to SEQ ID NO 4. In certain embodiments, the identity percentage is at least 55% or at least 60% or at least 65%, or at least 70% or at least 75%, or at least 80% or at least 85%, or at least 90% or at least 95%, or at least 99% or 100% identity to SEQ ID NO: 4.

The amino acid encoding the PB1 protein is characterized as having at least 72% identity to SEQ ID NO 6. In certain embodiments, the identity percentage is at least 75%, or at least 80% or at least 85%, or at least 90% or at least 95%, or at least 99% or 100% identity to SEQ ID NO: 6.

The amino acid encoding the PA protein is characterized as having at least 50% identity to SEQ ID NO 8. In certain embodiments, the identity is at least 55% or at least 60% or at least 65%, or at least 70% or at least 75%, or at least 80% or at least 85%, or at least 90% or at least 95%, or at least 99% or 100% identity to SEQ ID NO: 8.

The amino acid encoding the N protein is characterized as having at least 40% identity to SEQ ID NO 12. In certain embodiments, the identity is at least 45%, or at least 50% or at least 55% or at least 60% or at least 65%, or at least 70% or at least 75%, or at least 80% or at least 85%, or at least 90% or at least 95%, or at least 99% or 100% identity to SEQ ID NO: 12.

The amino acid encoding the NS1 protein is characterized as having at least 33% identity to SEQ ID NO 16. In certain embodiments, the identity is at least 40% or at least 45%, or at least 50% or at least 55%, at least 60% or at least 65%, at least 70% or at least 75%, at least 80% or at least 85%, at least 90% or at least 95%, at least 99% or 100% identity to SEQ ID NO: 16.

Influenza C viruses of the present invention can be propagated in cells, cell lines and host cells. Said cells, cell lines or host cells may be, for example, but not limited to, mammalian cells and non-mammalian cells, including insect and plant cells. Cells, cell lines and host cells in which influenza C viruses of the present invention may be propagated are readily known and accessible to those of ordinary skill in the art.

Influenza C viruses can be attenuated or inactivated prior to use in an immunogenic composition. Methods of attenuation and inactivation are well known to those skilled in the art. Methods for attenuation include, but are not limited to, serial passage in cell culture, ultraviolet irradiation, and chemical mutagenesis. Methods for inactivation include, but are not limited to, treatment with formalin, betapropriolactone (BPL) or binary ethyleneimine (BEI), or other methods known to those skilled in the art. Inactivation by formalin can be performed by mixing the virus suspension with 37% formaldehyde, to a final formaldehyde concentration of 0.05%. The virus-formaldehyde mixture is stirred constantly for approximately 24 hours at room temperature. The inactivated virus mixture is then tested for residual live virus by assaying for growth on a suitable cell line. Inactivation by BEI can be performed by mixing the virus suspension with 0.1 M BEI (2-bromo-ethylamine in 0.175 N NaOH), to a final BEI concentration of 1 mM. The virus-BEI mixture is stirred constantly for approximately 48 hours at room temperature, followed by the addition of 1.0 M sodium thiosulfate to a final concentration of 0.1 mM. Mixing is continued for an additional two hours. The inactivated virus mixture is tested for residual live virus by assaying for growth on a suitable cell line.

Immunogenic compositions of the present invention can include one or more veterinarily-acceptable carriers, such as any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others known to those skilled in the art. Stabilizers include albumin, among others known to the skilled artisan. Preservatives include merthiolate, among others known to the skilled artisan.

Immunogenic compositions of the present invention can include one or more adjuvants. Adjuvants include, but are not limited to, the RIBI adjuvant system (Ribi Inc.; Hamilton, Mont.), alum, aluminum hydroxide gel, oil-in water emulsions, water-in-oil emulsions such as, e.g., Freund's complete and incomplete adjuvants, Block co polymer (CytRx; Atlanta, Ga.), SAF-M (Chiron; Emeryville, Calif.), AMPHIGEN® adjuvant, saponin, Quil A, QS-21 (Cambridge Biotech Inc.; Cambridge, Mass.), GPI-0100 (Galenica Pharmaceuticals, Inc.; Birmingham, Ala.) or other saponin fractions, monophosphoryl lipid A, Avridine lipid-amine adjuvant, heat-labile enterotoxin from Escherichia coli (recombinant or otherwise), cholera toxin, or muramyl dipeptide, among many others known to those skilled in the art.

The amounts and concentrations of adjuvants and additives useful in the context of the present invention can readily be determined by the skilled artisan. In one embodiment, the present invention contemplates immunogenic compositions comprising from about 50 μg to about 2000 μg of adjuvant. In another embodiment, adjuvant is included in an amount from about 100 μg to about 1500 μg, or from about 250 μg to about 1000 μg, or from about 350 μg to about 750 μg. In another embodiment, adjuvant is included in an amount of about 500 μg/2 ml dose of the immunogenic composition.

A number of cytokines or lymphokines have been shown to have immune modulating activity, and thus may be used as adjuvants, including, but not limited to, the interleukins 1-α, 1-β, 2, 4, 5, 6, 7, 8, 10, 12 (see, e.g., U.S. Pat. No. 5,723,127), 13, 14, 15, 16, 17 and 18 (and its mutant forms), the interferons-α, β and γ, granulocyte-macrophage colony stimulating factor (see, e.g., U.S. Pat. No. 5,078,996 and ATCC Accession Number 39900), macrophage colony stimulating factor, granulocyte colony stimulating factor, GSF, and the tumor necrosis factors α and β. Still other adjuvants useful in this invention include a chemokine, including without limitation, MCP-1, MIP-1α, MIP-1β, and RANTES. Adhesion molecules, such as a selectin, e.g., L-selectin, P-selectin and E-selectin may also be useful as adjuvants. Still other useful adjuvants include, without limitation, a mucin-like molecule, e.g., CD34, GlyCAM-1 and MadCAM-1, a member of the integrin family such as LFA-1, VLA-1, Mac-1 and p150.95, a member of the immunoglobulin superfamily such as PECAM, ICAMs, e.g., ICAM-1, ICAM-2 and ICAM-3, CD2 and LFA-3, co-stimulatory molecules such as CD40 and CD40L, growth factors including vascular growth factor, nerve growth factor, fibroblast growth factor, epidermal growth factor, B7.2, PDGF, BL-1, and vascular endothelial growth factor, receptor molecules including Fas, TNF receptor, Flt, Apo-1, p55, WSL-1, DR3, TRAMP, Apo-3, AIR, LARD, NGRF, DR4, DRS, KILLER, TRAIL-R2, TRICK2, and DR6. Still another adjuvant molecule includes Caspase (ICE). See, also International Patent Publication Nos. WO98/17799 and WO99/43839, incorporated herein by reference.

Suitable adjuvants used to enhance an immune response include, without limitation, MPL™ (3-O-deacylated monophosphoryl lipid A; Corixa, Hamilton, Mont.), which is described in U.S. Pat. No. 4,912,094, which is hereby incorporated by reference. Also suitable for use as adjuvants are synthetic lipid A analogs or aminoalkyl glucosamine phosphate compounds (AGP), or derivatives or analogs thereof, which are available from Corixa (Hamilton, Mont.), and which are described in U.S. Pat. No. 6,113,918, which is hereby incorporated by reference. One such AGP is 2-[(R)-3-Tetradecanoyloxytetradecanoylamino] ethyl 2-Deoxy-4-O-phosphono-3-O-[(R)-3-tetradecanoyoxytetradecanoyl]-2-[(R)-3-tetradecanoyloxytetradecanoyl-amino]-b-D-glucopyranoside, which is also known as 529 (formerly known as RC529). This 529 adjuvant is formulated as an aqueous form or as a stable emulsion.

Still other adjuvants include mineral oil and water emulsions, aluminum salts (alum), such as aluminum hydroxide, aluminum phosphate, etc., Amphigen, Avridine, L121/squalene, D-lactide-polylactide/glycoside, pluronic polyols, muramyl dipeptide, killed Bordetella, saponins, such as Stimulon™ QS-21 (Antigenics, Framingham, Mass.), described in U.S. Pat. No. 5,057,540, which is hereby incorporated by reference, and particles generated therefrom such as ISCOMS (immunostimulating complexes), Mycobacterium tuberculosis, bacterial lipopolysaccharides, synthetic polynucleotides such as oligonucleotides containing a CpG motif (U.S. Pat. No. 6,207,646, which is hereby incorporated by reference), a pertussis toxin (PT), or an E. coli heat-labile toxin (LT), particularly LT-K63, LT-R72, PT-K9/G129; see, e.g., International Patent

Publication Nos. WO 93/13302 and WO 92/19265, incorporated herein by reference.

Also useful as adjuvants are cholera toxins and mutants thereof, including those described in published International Patent Application number WO 00/18434 (wherein the glutamic acid at amino acid position 29 is replaced by another amino acid, other than aspartic acid, preferably a histidine). Similar CT toxins or mutants are described in published International Patent Application number WO 02/098368 (wherein the isoleucine at amino acid position 16 is replaced by another amino acid, either alone or in combination with the replacement of the serine at amino acid position 68 by another amino acid; and/or wherein the valine at amino acid position 72 is replaced by another amino acid). Other CT toxins are described in published International Patent Application number WO 02/098369 (wherein the arginine at amino acid position 25 is replaced by another amino acid; and/or an amino acid is inserted at amino acid position 49; and/or two amino acids are inserted at amino acid positions 35 and 36).

The immunogenic compositions of the invention can also include surface-active substances. Suitable surface-active substances include, without limitation, quinone analogs, hexadecylamine, octadecylamine, octadecyl amino acid esters, lysolecithin, dimethyl-dioctadecylammonium bromide, methoxyhexadecylgylcerol, and pluronic polyols; polyamines, e.g., pyran, dextransulfate, poly IC, carbopol; peptides, e.g., muramyl peptide and dipeptide, dimethylglycine, tuftsin; oil emulsions; and mineral gels, e.g., aluminum phosphate, etc., and immune-stimulating complexes (ISCOMS).

The immunogenic compositions of the invention can also include antibiotics. Such antibiotics include, but are not limited to, those from the classes of aminoglycosides, carbapenems, cephalosporins, glycopeptides, macrolides, penicillins, polypeptides, quinolones, sulfonamides, and tetracyclines. In one embodiment, the present invention contemplates immunogenic compositions comprising from about 1 μg/ml to about 60 μg/ml of antibiotic. In another embodiment, the immunogenic compositions comprise from about 5 μg/ml to about 55 μg/ml of antibiotic, or from about 10 μg/ml to about 50 μg/ml of antibiotic, or from about 15 μg/ml to about 45 μg/ml of antibiotic, or from about 20 μg/ml to about 40 μg/ml of antibiotic, or from about 25 μg/ml to about 35 μg/ml of antibiotic. In yet another embodiment, the immunogenic compositions comprise less than about 30 μg/ml of antibiotic.

Other additives can also be included in the immunogenic compositions of this invention, including preservatives, stabilizing ingredients, and the like. Suitable exemplary preservatives include chlorobutanol, potassium sorbate, sorbic acid, sulfur dioxide, propyl gallate, the parabens, ethyl vanillin, glycerin, phenol, and parachlorophenol. Suitable stabilizing ingredients that may be used include, for example, casamino acids, sucrose, gelatin, phenol red, N-Z amine, monopotassium diphosphate, lactose, lactalbumin hydrolysate, and dried milk. The immunogenic compositions may also be incorporated into liposomes for use as an immunogenic composition, and may also contain other additives suitable for the selected mode of administration of the composition. The composition may include other pharmaceutically-acceptable excipients for developing powder, liquid or suspension dosage forms. See, e.g., Remington: The Science and Practice of Pharmacy, Vol. 2, 19^{th} edition (1995), e.g., Chapter 95 Aerosols; and International Patent Publication No. WO99/45966, the teachings of which are hereby incorporated by reference.

Immunogenic compositions of the invention can include other antigens. Antigens can be in the form of an inactivated whole or partial preparation of the microorganism, or in the form of antigenic molecules obtained by recombinant techniques or chemical synthesis. Other antigens appropriate for use in accordance with the present invention include, but are not limited to, those derived from pathogenic bacteria, such as Haemophilus somnus, Haemophilus parasuis, Bordetella bronchiseptica, Bacillus anthracis, Actinobacillus pleuropneumonie, Pasteurella multocida, Mannhemia haemolytica, Mycoplasma bovis, Mycobacterium bovis, Mycobacterium paratuberculosis, Clostridial spp., Streptococcus uberis, Staphylococcus aureus, Erysipelothrix rhusopathiae, Chlamydia spp., Brucella spp., Vibrio spp., Salmonella enterica serovars and Leptospira spp. Antigens can also be derived from pathogenic fungi, such as Candida, protozoa such as Cryptosporidium parvum, Neospora canium, Toxoplasma gondii, Eimeria spp., Babesia spp., Giardia spp., or helminths such as Ostertagia, Cooperia, Haemonchus, and Fasciola. Additional antigens can include pathogenic viruses, such as bovine coronavirus, bovine herpesviruses, bovine parainfluenza virus, bovine respiratory syncytial virus, bovine leukosis virus, rinderpest virus, foot and mouth disease virus, and rabies virus.

In other embodiments, the immunogenic composition may comprise purified amino acid sequences of the instant invention, as described above, including, without limitations SEQ ID NOs: 4, 6, 8, 10, 12, 14, 16-20.

### Forms, Dosages, Routes of Administration

Immunogenic compositions of the present invention can be administered to animals to induce an effective immune response against influenza C viruses. Accordingly, the present invention provides methods of stimulating an effective immune response against influenza C viruses by administering to an animal a therapeutically effective amount of an immunogenic composition of the present invention described herein.

Immunogenic compositions of the present invention can be made in various forms, depending upon the route of administration. For example, the immunogenic compositions can be made in the form of sterile aqueous solutions or dispersions suitable for injectable use, or made in lyophilized forms using freeze-drying techniques. Lyophilized immunogenic compositions are typically maintained at about 4° C., and can be reconstituted in a stabilizing solution, e.g., saline or HEPES, with or without adjuvant. Immunogenic compositions can also be made in the form of suspensions or emulsions.

These immunogenic compositions can contain additives suitable for administration via any conventional route of administration. The immunogenic compositions of the invention can be prepared for administration to subjects in the form of, for example, liquids, powders, aerosols, tablets, capsules, enteric-coated tablets or capsules, or suppositories. Thus, the immunogenic compositions may also include, but are not limited to, suspensions, solutions, emulsions in oily or aqueous vehicles, pastes, and implantable sustained-release or biodegradable formulations. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition. Other useful parenterally-administrable formulations include those which comprise the active ingredient in microcrystalline form, in a liposomal preparation, or as a component of a biodegradable polymer system. Compositions for sustained release or implantation may comprise pharmaceutically acceptable polymeric or hydrophobic materials, such as an emulsion, an ion exchange resin, a sparingly soluble polymer, or a sparingly soluble salt.

Immunogenic compositions of the present invention include a therapeutically effective amount of an influenza C virus. Purified viruses can be used directly in an immunogenic composition, or can be further attenuated, or inactivated. Typically, an immunogenic composition contains between about 1×10² and about 1×10¹² virus particles, or between about 1×10³ and about 1×10¹¹ virus particles, or between about 1×10⁴ and about 1×10¹⁰ virus particles, or between about 1×10⁵ and about 1×10⁹ virus particles, or between about 1×10⁶ and about 1×10⁸ virus particles. The precise amount of a virus in an immunogenic composition effective to provide a protective effect can be determined by a skilled artisan.

Immunogenic compositions generally comprise a veterinarily-acceptable carrier in a volume of between about 0.5 ml and about 5 ml. In another embodiment the volume of the carrier is between about 1 ml and about 4 ml, or between about 2 ml and about 3 ml. In another embodiment, the volume of the carrier is about 1 ml, or is about 2 ml, or is about 5 ml. Veterinarily-acceptable carriers suitable for use in immunogenic compositions can be any of those described herein.

Such carriers include, without limitation, water, saline, buffered saline, phosphate buffer, alcoholic/aqueous solutions, emulsions or suspensions. Other conventionally employed diluents, adjuvants and excipients, may be added in accordance with conventional techniques. Such carriers can include ethanol, polyols, and suitable mixtures thereof, vegetable oils, and injectable organic esters. Buffers and pH adjusting agents may also be employed. Buffers include, without limitation, salts prepared from an organic acid or base. Representative buffers include, without limitation, organic acid salts, such as salts of citric acid, e.g., citrates, ascorbic acid, gluconic acid, histidine-Hel, carbonic acid, tartaric acid, succinic acid, acetic acid, phthalic acid, Tris, trimethanmine hydrochloride, or phosphate buffers. Parenteral carriers can include sodium chloride solution, Ringer's dextrose, dextrose, trehalose, sucrose, lactated Ringer's, or fixed oils. Intravenous carriers can include fluid and nutrient replenishers, electrolyte replenishers, such as those based on Ringer's dextrose and the like. Preservatives and other additives such as, for example, antimicrobials, antioxidants, chelating agents (e.g., EDTA), inert gases and the like may also be provided in the pharmaceutical carriers. The present invention is not limited by the selection of the carrier. The preparation of these pharmaceutically acceptable compositions, from the above-described components, having appropriate pH, isotonicity, stability and other conventional characteristics, is within the skill of the art. See, e.g., texts such as Remington: The Science and Practice of Pharmacy, 20th ed, Lippincott Williams & Wilkins, pub., 2000; and The Handbook of Pharmaceutical Excipients, 4.sup.th edit., eds. R. C. Rowe et al, APhA Publications, 2003.

Those skilled in the art can readily determine whether a virus needs to be attenuated or inactivated before administration. In another embodiment of the present invention, an influenza C virus can be administered directly to an animal without additional attenuation. The amount of a virus that is therapeutically effective can vary depending on the particular virus used, the condition of the animal and/or the degree of infection, and can be determined by a skilled artisan.

In accordance with the methods of the present invention, a single dose can be administered to animals, or, alternatively, two or more inoculations can take place with intervals of from about two to about ten weeks. Boosting regimens can be required, and the dosage regimen can be adjusted to provide optimal immunization. Those skilled in the art can readily determine the optimal administration regimen.

Immunogenic compositions can be administered directly into the bloodstream, into muscle, or into an internal organ. Suitable means for parenteral administration include intravenous, intra-arterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques. Parenteral formulations are typically aqueous solutions which can contain excipients such as salts, carbohydrates and buffering agents (preferably to a pH of from about 3 to about 9, or from about 4 to about 8, or from about 5 to about 7.5, or from about 6 to about 7.5, or about 7 to about 7.5), but for some applications, they can be more suitably formulated as a sterile non-aqueous solution, or as a dried form to be used in conjunction with a suitable vehicle such as sterile, pyrogen-free water. The preparation of parenteral formulations under sterile conditions, for example, by lyophilisation, can readily be accomplished using standard pharmaceutical techniques well known to those skilled in the art.

The solubility of compounds used in the preparation of parenteral solutions can be increased by the use of appropriate formulation techniques known to the skilled artisan, such as the incorporation of solubility-enhancing agents including buffers, salts, surfactants, liposomes, cyclodextrins, and the like.

Formulations for parenteral administration can be formulated to be immediate and/or modified release. Modified release formulations include delayed, sustained, pulsed, controlled, targeted and programmed release. Thus compounds of the invention can be formulated as a solid, semi-solid, or thixotropic liquid for administration as an implanted depot, providing modified release of the active compound. Examples of such formulations include drug-coated stents and poly(dl-lactic-coglycolic)acid (PLGA) microspheres.

Immunogenic compositions of the present invention can also be administered topically to the skin or mucosa—that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions; liposomes can also be used. Typical carriers include alcohol, water, mineral oil, liquid petrolatum, white petrolatum, glycerin, polyethylene glycol and propylene glycol. Penetration enhancers can be incorporated; see, for example, Finnin and Morgan, J. Pharm Sci, 88 (10):955-958 (1999). Other means of topical administration include delivery by electroporation, iontophoresis, phonophoresis, sonophoresis and microneedle or needle-free (e.g., Powderject™, Bioject™, etc.) injection. Formulations for topical administration can be designed to be immediate and/or modified release. Modified release formulations include delayed, sustained, pulsed, controlled, targeted and programmed release.

Immunogenic compositions can also be administered intranasally or by inhalation, typically in the form of a dry powder (either alone or as a mixture, for example, in a dry blend with lactose, or as a mixed component particle, for example, mixed with phospholipids, such as phosphatidylcholine), from a dry powder inhaler, or as an aerosol spray from a pressurized container, pump, spray, atomizer (preferably an atomizer using electrohydrodynamics to produce a fine mist). It can also be administered via a nebulizer, with or without the use of a suitable propellant, such as 1,1,1,2-tetrafluoroethane or 1,1,1,2,3,3,3-heptafluoropropane. For intranasal use, the powder can comprise a bioadhesive agent, for example, chitosan or cyclodextrin. The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is generally micronized to a size suitable for delivery by inhalation (typically less than about 5 microns). This can be achieved by any appropriate comminuting method, such as spiral jet milling, fluid bed jet milling, supercritical fluid processing (to form nanoparticles), high pressure homogenization, or spray drying.

Capsules (made, for example, from gelatin or hydroxypropylmethylcellulose), blisters, and cartridges for use in an inhaler or insufflators, can be formulated to contain a powder mix of the compound of the invention. A suitable powder base could be lactose or starch, and a performance modifier could be 1-leucine, mannitol, or magnesium stearate. The lactose can be anhydrous, or in the form of the monohydrate. Other suitable excipients include dextran, glucose, maltose, sorbitol, xylitol, fructose, sucrose and trehalose.

A suitable solution formulation for use in an atomizer, using electrohydrodynamics to produce a fine mist, can contain from about 1 μg to about 20 mg of the compound of the invention per actuation, and the actuation volume can vary from about 1 μl to about 100 μl. In another embodiment, the amount of compound per actuation can range from about 100 μg to about 15 mg, or from about 500 μg to about 10 mg, or from about 1 mg to about 10 mg, or from about 2.5 μg to about 5 mg. In another embodiment, the actuation volume can range from about 5 μl to about 75 μl, or from about 10 μl to about 50 μl, or from about 15 μl to about 25 μl. A typical formulation can comprise the compound of the invention, propylene glycol, sterile water, ethanol and sodium chloride. Alternative solvents which can be used instead of propylene glycol include glycerol and polyethylene glycol.

Formulations for inhaled/intranasal administration can be formulated to be immediate and/or modified release using, for example, PLGA. Modified release formulations include delayed, sustained, pulsed, controlled, targeted and programmed release.

In the case of dry powder inhalers and aerosols, the dosage unit is generally determined by means of a valve which delivers a metered amount. Units in accordance with the invention are typically arranged to administer a metered dose or “puff” containing from about 10 ng to about 100 μg of the compound of the invention. In another embodiment, the amount of compound administered in a metered dose is from about 50 ng to about 75 μg, or from about 100 ng to about 50 μg, or from about 500 ng to about 25 μg, or from about 750 ng to about 10 μg, or from about 1 μg to about 5 μg. The overall daily dose will typically be in the range from about 1 μg to about 100 mg, which can be administered in a single dose or, more usually, as divided doses throughout the day. In another embodiment, the overall daily dose can range from about 50 μg to about 75 mg, or from about 100 μg to about 50 mg, or from about 500 μg to about 25 mg, or from about 750 μg to about 10 mg, or from about 1 mg to about 5 mg.

Immunogenic compositions of the present invention can also be administered orally or perorally—that is, into a subject's body through or by way of the mouth, and involves swallowing or transport through the oral mucosa (e.g., sublingual or buccal absorption, or both). Suitable flavors, such as menthol and levomenthol, or sweeteners, such as saccharin or saccharin sodium, can be added to those formulations of the invention intended for oral or peroral administration.

Immunogenic compositions of the present invention can be administered rectally or vaginally, for example, in the form of a suppository, pessary, or enema. Cocoa butter is a traditional suppository base, but various alternatives can be used as appropriate. Formulations for rectal/vaginal administration can be formulated to be immediate and/or modified release. Modified release formulations include delayed, sustained, pulsed, controlled, targeted and programmed release.

Immunogenic compositions of the present invention can also be administered directly to the eye or ear, typically in the form of drops of a micronized suspension, or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, can be incorporated together with a preservative, such as benzalkonium chloride. Such formulations can also be delivered by iontophoresis. Formulations for ocular/aural administration can be formulated to be immediate and/or modified release. Modified release formulations include delayed, sustained, pulsed, controlled, targeted and programmed release.

The immunogenic compositions of the present invention are not limited by the selection of the conventional, physiologically-acceptable carriers, adjuvants, or other ingredients useful in pharmaceutical preparations of the types described above. The preparation of these pharmaceutically acceptable compositions, from the above-described components, having appropriate pH isotonicity, stability and other conventional characteristics, is within the skill of the art.

In general, selection of the appropriate “effective amount” or dosage for the components of the immunogenic compositions of the present invention will also be based upon the identity of the antigen in the immunogenic composition(s) employed, as well as the physical condition of the subject, most especially including the general health, age and weight of the immunized subject. The method and routes of administration, and the presence of additional components in the immunogenic compositions, may also affect the dosages and amounts of the compositions. Such selection, and upward or downward adjustment of the effective dose, is within the skill of the art. The amount of composition required to induce an immune response, preferably a protective response, or produce an exogenous effect in the subject without significant adverse side effects, varies depending upon these factors. Suitable doses are readily determined by persons skilled in the art.

Similarly, the order of immunogenic composition administration and the time periods between individual administrations may be selected by one of skill in the art based upon the physical characteristics and precise responses of the host to the application of the method. Such optimization is expected to be well within the skill of the art.

The immunogenic compositions of the present invention can also be used in the preparation of a medicament for treating and/or preventing influenzavirus C infection in an animal.

### Recombinant Techniques

In yet other embodiments of the invention, the immunogenic composition may comprise a recombinant vaccine. Such recombinant vaccines would generally comprise a vector and a heterologous insert comprising an antigen. The heterologous inserts in some embodiments comprise one or more nucleic acid sequences encoding the amino acid sequences of the instant invention, as described above, e.g., SEQ ID NOs: 4, 6, 8, 10, 12, 14, 16-20. The insert may optionally comprise a heterologous promoter, such as, for example, synthetic promoters known in the art. Alternatively, the promoters of the host vector may exert transcriptional control over the expression of the inserts. Suitable non-limiting examples of promoters (which may be native or heterologous, depending on the choice of the vector) are H6 vaccinia promoter, I3L vaccinia promoter, 42K poxviral promoter, 7.5K vaccinia promoter, and Pi vaccinia promoter.

Suitable vectors have been described elsewhere in this application. In some embodiments, the vectors may be viral vectors, including, without limitations, vaccinia and pox viral vectors, such as parapox, racoonpox, swinepox, and different avipox vectors (e.g., canarypox and fowlpox strains). Currently contemplated viral strains include D1701, ALVAC, TROVAC, NYVAC strains. Generally, sequences that are non-essential for the viral host are suitable insertions sites for the inserts of the instant invention. The strains recited above are well-characterized in the art and some insertions sites in these vectors are well known. See, e.g., U.S. Pat. No. 5,174,993; U.S. Pat No. 5,505,941 U.S. Pat. No. 5,766,599 U.S. Pat. No. 5,756,103, U.S. Pat. No. 7,638,134, U.S. Pat. No. 6,365,393.

There are several known methods or techniques that can be used to clone and express the nucleotide sequences of the present invention. For example, the sequences can be isolated as restriction fragments and cloned into cloning and/or expression vectors. The sequences can also be PCR amplified and cloned into cloning and/or expression vectors, or they can be cloned by a combination of these two methods. Standard molecular biology techniques known in the art, and not specifically described, can be generally followed as described in Sambrook et al., Molecular Cloning: A Laboratory Manual, Cold Spring Harbor Laboratory Press, New York (1989); Ausubel et al., Current Protocols in Molecular Biology, John Wiley and Sons, Baltimore, Md. (1989); Perbal, A Practical Guide to Molecular Cloning, John Wiley & Sons, New York (1988); Watson et al., Recombinant DNA, Scientific American Books, New York; Birren et al (eds) Genome Analysis: A Laboratory Manual Series, Vols. 1-4 Cold Spring Harbor Laboratory Press, New York (1998); and methodology set forth in U.S. Pat. Nos. 4,666,828; 4,683,202; 4,801,531; 5,192,659 and 5,272,057. Polymerase chain reaction (PCR) is carried out generally as described in PCR Protocols: A Guide to Methods and Applications, Academic Press, San Diego, Calif. (1990).

The present invention encompasses the use of prokaryotic and eukaryotic expression systems, including vectors and host cells, which may be used to express both truncated and full-length forms of the recombinant polypeptides expressed by the nucleotide sequences of the present invention. A variety of host-expression vector systems may be utilized to express the polypeptides of the present invention. Such host-expression systems also represent vehicles by which the coding sequences of interest may be cloned and subsequently purified. The present invention further provides for host cells which may, when transformed or transfected with the appropriate vector or nucleotide sequence, express the encoded polypeptide gene product of the invention. Such host cells include, but are not limited to, microorganisms such as bacteria (e.g., E. coli, B. subtilis) transformed with recombinant bacteriophage DNA, plasmid DNA or cosmid DNA expression vectors containing coding sequences; yeast (e.g., Saccharomyces, Pichia) transformed with recombinant yeast expression vectors containing the gene product coding sequences; insect cell systems infected with recombinant virus expression vectors (e.g., baculovirus) containing the coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

Generally, the vectors of the invention can be derived from, but not limited to, bacterial plasmids, from bacteriophage, from yeast episomes, from yeast chromosomal elements, from viruses (e.g., as described above), from mammalian chromosomes, and from combinations thereof, such as those derived from plasmid and bacteriophage genetic elements including, but not limited to, cosmids and phagemids.

Vectors of the present invention can be used for the expression of polypeptides. Generally, the vectors of the invention include cis-acting regulatory regions operably linked to the polynucleotide that encodes the polypeptides to be expressed. The regulatory regions may be constitutive or inducible. Appropriate trans-acting factors are supplied by the host by an in vitro translation system, by a complementing vector, or by the vector itself upon introduction into the host.

The vectors of the invention can include any elements typically included in an expression or display vector, including, but not limited to, origin of replication sequences, one or more promoters, antibiotic resistance genes, leader or signal peptide sequences, various tag sequences, stuffer sequences that may encode a gene whose polypeptide confers antibiotic resistance, restriction sites, ribosome binding sites, translational enhancers (sequences capable of forming stem loop structures for mRNA stability post-transcription), sequences that encode amino acids lacking a stop codon, and sequences that encode a bacterial coat protein.

### Detection, Diagnostic Methods

The present invention provides methods of diagnosing infection by a bovine influenza C virus in an animal. This diagnosis can be accomplished via any of various diagnostic methods, including but not limited to ELISA, Western blotting, PCR, nucleic acid-based assays, including Southern or Northern blot analysis, and sequencing. Alternatively, protein-based assays can be employed. In protein-based assays, cells or tissues suspected of being infected can be isolated from the animal. Cellular extracts can be made from such cells or tissues, and can be subjected to, e.g., Western Blot, using appropriate antibodies that can distinctively detect the presence of the virus.

The extent and nature of the immune responses induced in the animal can be assessed by using a variety of techniques. For example, sera can be collected from the inoculated animals, and tested for the presence or absence of antibodies specific for the virus, e.g., in a conventional virus neutralization assay. Detection of responding cytotoxic T-lymphocytes (CTLs) in lymphoid tissues can be achieved by assays such as T cell proliferation, which is indicative of the induction of a cellular immune response. The relevant techniques are well described in the art, e.g., Coligan et al. Current Protocols in Immunology, John Wiley & Sons Inc. (1994).

### Kits

Inasmuch as it may be desirable to administer an immunogenic composition individually or in combination with additional compounds—for example, for the purpose of treating a particular disease or condition—it is within the scope of the present invention that an immunogenic composition can conveniently be included in, or combined in, the form of a kit suitable for administration or co-administration of the compositions. Kits of the present invention can comprise one or more separate pharmaceutical compositions, at least one of which is an immunogenic composition in accordance with the present invention, and a means for separately retaining said compositions, such as containers, a divided bottle, or a divided foil packet. An example of such a kit is a syringe and needle, and the like. A kit of the present invention is particularly suitable for administering different dosage forms, for example, oral or parenteral, for administering the separate compositions at different dosage intervals, or for titrating the separate compositions against one another. To assist one administering a composition of the present invention, the kit typically comprises directions for administration.

Another kit of the present invention can comprise one or more reagents useful for the detection of an influenza C virus. The kit can include reagents for analyzing a sample for the presence of whole influenza C virus, or influenza C virus polypeptides, epitopes or polynucleotide sequences. In certain embodiments, the kits can include a set of printed instructions or a label indicating that the kit is useful for the detection of animals infected with influenza C virus.

### Antibody, Antibodies

Antibodies can either be monoclonal, polyclonal, or recombinant. The antibodies can be prepared against the immunogen or a portion thereof. For example, a synthetic peptide based on the amino acid sequence of the immunogen, or prepared recombinantly by cloning techniques, or the natural gene product and/or portions thereof, can be isolated and used as the immunogen. Immunogens can be used to produce antibodies by standard antibody production technology well known to those skilled in the art, such as described generally in Harlow and Lane, “Antibodies: A Laboratory Manual”, Cold Spring Harbor Laboratory, Cold Spring Harbor, N.Y., (1988), and Borrebaeck, “Antibody Engineering—A Practical Guide”, W. H. Freeman and Co. (1992). Antibody fragments can also be prepared from the antibodies, and include Fab, F(ab′)₂, and Fv, by methods known to those skilled in the art.

Suitable non-limiting examples of immunogens include proteins or protein fragments of bovine influenza C virus, such as, e.g., SEQ ID NOs: 4, 6, 8, 10, 12, 14, 16-20.

In one embodiment of the present invention the antibody of the invention further provides an intact immunoglobulin capable of specific binding to a target, such as a carbohydrate, polynucleotide, lipid, polypeptide, etc., through at least one antigen recognition site located in the variable region of the immunoglobulin molecule. An intact antibody has two light and two heavy chains. Thus a single isolated intact antibody may be a polyclonal antibody, a monoclonal antibody, a synthetic antibody, a recombinant antibody, a chimeric antibody, or a heterochimeric antibody.

In the production of antibodies, screening for the desired antibody can be accomplished by standard methods in immunology known in the art. Techniques not specifically described are generally followed as in Stites, et al. (eds), “Basic and Clinical Immunology” (8th Edition), Appleton and Lange, Norwalk, Conn. (1994), and Mishell and Shiigi (eds), “Selected Methods in Cellular Immunology”, W. H. Freeman and Co., New York (1980). In general, ELISAs and Western blotting are the preferred types of immunoassays; both assays are well known to those skilled in the art. Both polyclonal and monoclonal antibodies can be used in the assays. The antibody can be bound to a solid support substrate, or conjugated with a detectable moiety, or be both bound and conjugated as is well known in the art. (For a general discussion of conjugation of fluorescent or enzymatic moieties, see Johnstone and Thorpe, “Immunochemistry in Practice”, Blackwell Scientific Publications, Oxford (1982).) The binding of antibodies to a solid support substrate is also well known in the art. (For a general discussion, see Harlow and Lane (1988), and Borrebaeck (1992).) The detectable moieties contemplated for use in the present invention can include, but are not limited to, fluorescent, metallic, enzymatic and radioactive markers such as biotin, gold, ferritin, alkaline phosphatase, b-galactosidase, peroxidase, urease, fluorescein, rhodamine, tritium, ¹⁴C and iodination.

The present invention is further illustrated by, but by no means limited to, the following examples.

### EXAMPLES

### Example 1

### Virus Isolation

A pool of nasal swabs from a bovine respiratory disease case was used for virus isolation. Primary isolation of the virus was done on HRT-18G cells. Subsequent passaging on HRT-18G cells resulted in a larger preparation of high titer virus (˜1×10⁸ TCID₅₀/mL), which was used in later studies. Cytopathic effect (CPE) was not observed on the infected HRT-18G cells; however, giant cells were occasionally present, indicative of virus-induced cell fusion. The presence of virus was confirmed by indirect immunofluorescent antibody (IFA) staining, using pooled convalescent cattle serum as a source of primary antibody, followed by anti-bovine FITC-conjugated secondary antibody. The virus was initially identified as Isolate #14. Extraneous viral pathogens of bovine origin were not identified in the culture fluids.

### Example 2

### Susceptibility of Various Cell Types

Various cell lines were evaluated for their susceptibility to the isolated virus. Virus titrations were also performed on the culture fluids harvested from various productive cell infections. CPE was generally not observed, except on NLST (swine testicular) cells.

TABLE 1
Cell lines tested for virus growth, and yields.

| Cell Type | Treatment | Log₁₀ TCID₅₀/mL |
|---|---|---|
| HRT-18G | Trypsin | 8.2 |
| HRT-18G | None | 8.3 |
| BK-6 | Trypsin | 7.5 |
| BK-6 | None | 8.0 |
| NLST | Trypsin | 7.5 |
| NLST | None | 7.7 |
| MDBK | Trypsin | 7.2 |
| MDBK | None | 7.3 |
| BT/CS | Trypsin | 7.5 |
| BT/CS | None | 7.0 |
| CRFK | Trypsin | 5.8 |
| CRFK | None | 7.3 |
| MA104 | Trypsin | 6.5 |
| MA104 | None | 6.5 |
| MDCK | Trypsin | 6.5 |
| MDCK | None | 6.2 |
| NLED5 | Trypsin | 5.8 |
| NLED5 | None | 6.7 |
| Vero | Trypsin | 5.7 |
| Vero | None | 5.2 |
| NLFK | Trypsin | 5.0 |
| NLFK | None | 5.7 |
| NLDK | Trypsin | 5.0 |
| NLDK | None | 5.2 |

Trypsin treatment did not appear to be necessary for propagation of the virus. Also, upon further investigation, it was concluded that though the virus did infect some cell lines, it was not a productive infection (sometimes referred to as “aborted infection”). HRT-18G, NLST, MA-104 and BK-6 cells were productively infected, with HRT-18G and BK-6 cells yielding the highest titers.

### Example 3

### Hemagglutination Assay

A standard hemagglutination (HA) test was performed, using red blood cells from a rooster, as well as from a guinea pig. Results (not shown) indicated that the bovine virus was HA positive, and yielded high titers in the HA test.

### Example 4

### Electron Microscopy

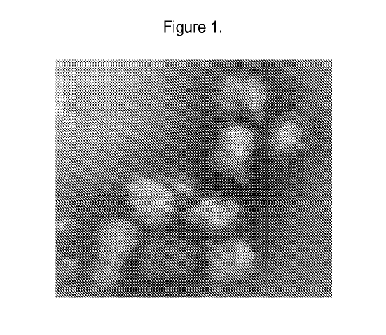

Cell culture fluids containing the virus were clarified, and the virus was concentrated. The pelleted virus was subjected to sodium phosphotungstate staining. Following this treatment, the preparation was observed under an electron microscope. Negative-stained images of the virus showed enveloped, spiked, polymorphic viral particles, ˜100 nm in diameter (FIG. 1).

### Example 5

### Amplification of a Polymerase Gene by RT-PCR

Total RNA was isolated from culture fluids infected with virus using a Viral RNA Mini Kit (Qiagen; Valencia, Calif.). A universal primer pair, designed for amplifying a paramyxovirus, was synthesized: Par F1 (GAA GGI TAT TGT CAI AAR NTN TGG AC) and Par R (GCT GAA GTT ACI GGI TCI CCD ATR TTN C). SuperScript® III One-Step RT-PCR System with Platinum® Taq (Life Technologies; Grand Island, N.Y.) was used to set up the RT-PCR reactions, with the only variance being that a lower annealing temperature (44° C.) was used. A PCR product of 448 nt fragment was generated, separated on an E-gel (Life Technologies), and extracted from the agarose for sequencing. The Par F1 and Par R primers were used as sequencing primers. The DNA sequence obtained was translated, and used as a query sequence to search for related sequences in GenBank. The hit with the highest protein identity (57%) was a polymerase basic protein (PB2) from human influenza C virus.

### Example 6

### Virus Sequence Determination

Cell culture fluids were harvested and clarified, and virus was concentrated by ultracentrifugation. Viral RNA was extracted using a Viral RNA Mini Kit (Qiagen). Quality assessment was performed on two RNA samples (RNA-1; RNA-2) using NanoDrop spectroscopy (Thermo Scientific; Wilmington, Del.). Sequencing technology from Illumina (San Diego, Calif.) was employed. RNA samples (10.5 μl) were used for library preparation. RNA was fragmented, and cDNA libraries were constructed from the RNA using TruSeq mRNA Sample Prep Kit (Illumina). The libraries were bar-coded using the standard Illumina adaptors. The resulting final libraries were purified, and size selected using Agencort AMpure XP beads (Beckman Coulter: Beverly, Mass.) and agarose gel electrophoresis. Libraries from replicate samples were determined to have an average fragment size of 280 bp. The libraries were quantified using the KAPA Library Quantification Kit (Kapa Biosystems; Woburn, Mass.).

Extensive qPCR evaluation was performed in order to accurately measure the amounts of input DNA, in order to facilitate optimum cluster generation during progressive cycles of thermal amplification on the cBOT platform (Illumina). In order to generate clusters of DNA on the cBOT, the libraries were pooled and loaded at different concentrations, ranging from 4 to 8 pmol, along with reagents from the cBOT Paired-End Cluster Generation Kit (Illumina). Sequencing templates were immobilized on a proprietary flow cell surface, and samples were loaded onto the Illumina Genome Analyzer to generate paired end reads of 150 bases (2×150 bp run). A successful run generated densities on the order of ten million single-molecule clusters for each sample. Using Bowtie 2 (Langmead and Salzberg; Nature Methods, 9:357-359, 2012), reads mapping to the human genome were identified and removed. 13% of the RNA-1 reads, and 11% of RNA-2 reads, mapped to the human genome. After filtering, 4.6 million reads remained for RNA-1, and 1.9 million reads for RNA-2. With an expected genome size of 15-20 kb, this gives a coverage of 21,000X-28,000X for RNA-1, and 9,000X -11,000X for RNA-2.

Reads were assembled into contigs using ABySS (Simpson et al; Genome Res., 19:1117-1123, 2009). For RNA-1, 10 contigs with a total size of 13.8 kb were generated. For RNA-2, 16 contigs with a total size of 14.7 kb were generated. These contigs were annotated using BlastX (Altschul et al., J Mol Biol., 215(3):403-410, 1990), searching against the GenBank non-redundant protein database. The larger contigs had strong homology to proteins from human influenza C virus. The human influenza C virus is a negative-sense, segmented RNA virus. Its genome contains seven RNA segments, which encode for the following proteins (listed in order of decreasing MW): viral polymerases PB2, PB1, and PA; hemagglutinin-neuraminidase (HE), nucleocapsid (N), matrix (M), and non-structural protein, NS1.

TABLE 2
Contigs and their Identity Comparison to the Human Influenza C Virus.

| | | | Influenza | % Identity to |
|---|---|---|---|---|
| | | | C Virus | GenBank Protein |
| SEQ ID NOs | | Amino | Encoded | Sequence of human |
| (nuclecic/amino) | Nucleotides | Acids | Proteins | influenza C virus |
| 3/4 | 2330 | 744 | PB2 | 52 |
| 5/6 | 2284 | 745 | PB1 | 72 |
| 7/8 | 2179 | 710 | PA | 50 |
| 9/10 | 2095 | 664 | HE | 54 |
| 11/12 | 1753 | 552 | N | 40 |
| 13/14 | 1203 | 387 | M | 36 |
| 15/16 | 852 | 248 | NS1 | 33 |

### Example 7

### Determination of Conserved Fragments of Bovine C Influenza Genome

Since, in general, conserved sequences have functional significance, additional Blast searches were performed to determine uniqueness of shorter fragments of bovine C Influenza genome that are conserved between bovine and human influenza C viruses. M protein and HA protein were used as representative examples.

TABLE 3
BLAST settings.

| Parameter | Value |
|---|---|
| Algorithm | Pblast (protein-protein BLAST) |
| Database | Non-redundant protein sequences |
| Max target sequences | 100 |
| Automatic adjustment for short | Yes |
| queries? | |
| Expect threshold | 10 |
| Word Size | 3 |
| Max Matches in a query | 0 |
| Matrix | BLOSUM62 |
| Match/Mismatch score | 1/−2 |
| Gap Costs | Existence 11; Extension 1 |
| Compositional adjustment non-default | Conditional compositional score |
| value | matrix adjustment |
| Low complexity regions filter | None |
| Species specific repeats filter | None |
| Masks (both lookup table and lower | None |
| case letters) | |
| Template length | None |
| Template Type | Coding |

Using the BLAST settings described in Table 5, the applicants have surprisingly discovered that amino acids 145-181 of the M protein of bovine influenza C virus (SEQ ID NO: 17) was 67% identical and 92% similar (33/36 positives) to human influenza C virus Saitama strain (C/Saitama/2/2000). Identity and positive match value to all other nucleic acid sequences was the same or even lower. The other non-overlapping regions of bovine Influenza C virus were even less positively matched than any sequence in the database.

When Applicants analyzed HE protein (SEQ ID NO: 10), they have discovered that two amino acid regions, amino acids 58-136 (SEQ ID NO: 18) and amino acids 539-598 (SEQ ID NO: 19) were highly positively matched to human influenza C virus Ann Arbor strain (C/Ann Arbor/1/50). When each of these sequences was analyzed separately, it was surprisingly discovered that each of them was unique: SEQ ID NO: 18 was 78% identical and 84% positively matched (Positives 66/79 (84%)) to human influenza C virus strain Milan (C/Milan/1101/2009). SEQ ID NO: 19 was 63% identical and 85% positively matched (positives 51/60 (85%)) to human influenza C Alberta strain (C/Alberta/3502/2011). Even more surprisingly, SEQ ID NO: 20 (amino acids 36-79 of SEQ ID NO: 18) was 93.1% (41/44) identical to human influenza C virus strain Milan (C/Milan/1101/2009).

### Example 8

### Animal Studies

Calf study #1. A single cesarean-derived 3-day old calf was inoculated with 10 mL of clarified viral culture fluids intranasally. Following challenge, nasal and fecal swabs were collected daily, and clinical observations were recorded. Virus isolations were subsequently performed on the nasal and fecal swabs, using HRT-18G cells. Virus isolations were also performed on various lung tissue samples. Bovine influenza C virus was isolated from nasal swabs from study day 1 through day 7, and also isolated from lung samples (Table 4). However, virus was not isolated from the fecal swabs.

TABLE 4
Bovine influenza C virus isolation results from Calf study #1.

| Study Day | Sample Type | Titers in Log₁₀ TCID₅₀/mL |
|---|---|---|
| 1 | Nasal swab | <1.5 |
| 2 | Nasal swab | 3.0 |
| 3 | Nasal swab | 5.5 |
| 4 | Nasal swab | 5.3 |
| 5 | Nasal swab | 5.5 |
| 6 | Nasal swab | 4.8 |
| 7 | Nasal swab | 2.8 |
| 1 | Fecal Swab | <1.5 |
| 7 | Fecal Swab | <1.5 |
| 7 | Right ant. lobe | 5.0 |
| 7 | Left ant. lobe | 5.8 |
| 7 | Right caudal lobe | 4.0 |
| 7 | Left caudal lobe | 4.5 |
| 7 | Right middle lobe | 6.3 |
| 7 | Left middle lobe | 6.3 |
| 7 | Accessory tissue | 4.3 |
| 7 | Trachea | 5.3 |
| | Inoculum | 8.3 |

There were no observed clinical symptoms following inoculation of the calf with clarified culture fluids. At necropsy, no significant gross pathological changes were identified. Histopathologic evaluation indicated that the lungs exhibited mild interstitial pneumonia, consistent with viral infection. These mild lesions would not be expected to be seen grossly, however.

Calf study #2. A single cesarean-derived 3-day old calf was inoculated by intranasal administration with 10 mL of clarified pooled samples (nasal swabs and lung homogenates) collected from the calf in Study #1. Following challenge, nasal and fecal swabs were collected daily, and clinical observations were recorded. Virus isolations were subsequently performed on the nasal and fecal swabs, using HRT-18G cells. Virus isolations were also performed on various lung tissue samples. Bovine influenza C virus was isolated from nasal swabs from study day 1 through day 6, as well as from lung samples (Table 5). Once again, no virus was isolated from the fecal swabs.

TABLE 5
Bovine Influenza C Virus Isolation Results from Calf Study #2.

| Study Day | Sample Type | IFA | Virus Titers |
|---|---|---|---|
| 0 | Nasal swab | Negative | <1.5 |
| 1 | Nasal swab | Positive | 1.7 |
| 2 | Nasal swab | Positive | 5.2 |
| 3 | Nasal swab | Positive | 2.2 |
| 4 | Nasal swab | Positive | 3.8 |
| 5 | Nasal swab | Positive | 3.5 |
| 6 | Nasal swab | Positive | 2.6 |
| 7 | Nasal swab | Negative | <1.5 |
| 8 | Nasal swab | Negative | <1.6 |
| 0 | Fecal Swab | Negative | <1.5 |
| 1 | Fecal Swab | Negative | <1.5 |
| 8 | Fecal Swab | Negative | <1.5 |
| 8 | Right cranial lung | Positive | ND |
| 8 | Left cranial lung | Negative | ND |
| 8 | Right caudal lung | Positive | ND |
| 8 | Left caudal lung | Positive | ND |
| 8 | Right middle lung | Positive | ND |
| 8 | Left middle lung | Positive | ND |
| 8 | Accessory tissue | Positive | ND |
| 8 | Trachea | Negative | 2.5 |
| 8 | Trach/bronch. | Negative | ND |
| 8 | Lung lavage | Positive | 2.5 |

ND = Not Determined

There were no observed clinical symptoms following inoculation of the calf with pooled samples from the previous study. At necropsy, no significant gross pathological changes were identified. Lung samples had mild to moderate multifocal broncho-interstitial histiocytic pneumonia, with intralesional intracytoplasimic eosinophilic inclusion bodies, and a few syncytial cells.

### Example 9

### Seroepidemiological Survey

Serum samples from various herds/locations were evaluated for the presence of antibody to bovine influenza C virus, using a direct fluorescent antibody assay (FA). HRT-18G cells were infected with the virus, and following incubation, were fixed in 80% acetone. Serum samples were diluted in PBS, starting at 1:40, followed by 4-fold serial dilutions. Diluted serum samples were added to the wells of fixed cells, and incubated for 40-60 minutes. After multiple washes, goat-anti bovine FITC-conjugated antibody was added, and incubated for 30 minutes. Plates were washed and read under a UV light source. Titers were reported as the reciprocal of the highest dilution that was FA positive. Results from various herds sampled are listed below:

Herd C-1. 182 serum samples (collected in Summer 2011) were tested; 85 had titers greater than or equal to 40. The prevalence of anti-bovine influenza C virus antibodies was 47.8%. Twenty positive samples had titers of 2560, indicating an active infection.

Herd C-2. 339 serum samples were collected from the same herd (C-1), but 2-3 months earlier. Twenty-four of 160 (15%) samples were positive by FA. Out of these positive samples, 2 had titers of 2560, indicating an active infection.

Herd D. 173 bovine serum samples were collected; 4 (2.3%) had antibody titers of 80 -320 to bovine influenza C virus, suggesting that the antibody may have been maternally-derived, and that these weaning-age calves likely had not been exposed to the virus.

Herd X. 72 serum samples were collected from calves on a research farm; 11 (15.2%) had FA titers greater than 640, with the exception of one (FA titer of 160).

1.-7. (canceled)

8. An immunogenic composition comprising an adjuvant and at least one of:
a) an isolated bovine influenza C virus:,
b) an isolated nucleic acid comprising one or more of the nucleic acids; a nucleic acid encoding a protein having greater than 52% identity to SEQ ID NO: 4; a nucleic acid encoding a protein having greater than 72% identity to SEQ ID NO: 6; a nucleic acid encoding a protein having greater than 50% identity to SEQ ID NO: 8; a nucleic acid encoding a protein having greater than 54% identity to SEQ ID NO: 10; a nucleic acid encoding a protein having greater than 40% identity to SEQ ID NO: 12; a nucleic acid encoding a protein having greater than 36% identity to SEQ ID NO: 14; and a nucleic acid encoding a protein having greater than 33% identity to SEQ ID NO: 16, wherein SEQ ID NO:4 encodes an influenza C “PB2” protein; SEQ ID NO:6 encodes an influenza C “PB1” protein; SEQ ID NO; 8 encodes an influenza “PA” protein; SEQ ID NO:10 encodes an influenza C “HE” protein; SEQ ID NO:12 encodes an influenza C “N” protein; SEQ ID NO:14 encodes an influenza C “M” protein; and SEQ ID NO;16 encodes an influenza C “NS1” protein;
c) one or more isolated polypeptides selected from the group consisting of: the influenza C PB2 protein of SEQ ID NO: 4; the PB1 protein of SEQ ID NO: 8; the HE protein of SEQ ID NO: 10; the N protein of SEQ ID NO: 12; the M protein of SEQ ID NO 14: and the NS1 protein of SEQ ID NO: 16;
d) any combination thereof.

9. The composition of claim 8, wherein the adjuvant is selected from the group consisting of RIBI adjuvant system, alum, aluminum hydroxide gel, oil-in water emulsions, water-in-oil emulsions Block co polymer, SAF-M (Chiron; Emeryville, Calf.), saponin, Quil A, QS-21, GPI-0100, monophosphoryl lipid A, Avridine, heat-labile enterotoxin from Escherichia coli), cholera toxin, muramyl dipeptide, ISCOMS (immunostimulating complexes), bacterial lipopolysaccharides, as CpG oligonucleotides and any combination thereof.

10. An expression vector comprising one or more polynucleotides selected from the group consisting of: a nucleic acid encoding a protein having greater than 52% identity to SEQ ID NO: 4; a nucleic acid having greater than 72% identity to SEQ ID NO: 6; a nucleic acid encoding a protein having greater than 50% identity to SEQ ID NO: 8; a nucleic acid encoding a protein having greater than 54% identity to SEQ ID NO: 10; a nucleic acid encoding a protein having greater than 36% identity to SEQ ID NO: 12; a nucleic acid encoding a protein having greater than 36% identity to SEQ ID NO: 14; and a nucleic acid encoding a protein having greater than 33% identity to SEQ ID NO: 16.

11. The expression vector of claim 10 comprising one or more of SEQ ID NO:' s 3, 5, 7, 9, 11, 13, and 15.

12. (canceled)

13. (canceled)

14. (canceled)

15. (canceled)

16. The immunogenic composition of claim 8, wherein said virus is inactivated, live or live attenuated.

17. A method of treating or protecting an animal from disease caused by an influenza C virus, the method comprising administering to the animal the immunogenic composition of claim 8.

18. A method of detecting exposure of an animal to influenza C virus that comprises determining the presence of any one or more of SEQ ID NO:'s 4-16 in a sample from the animal.

19. An antibody that specifically binds to the isolated influenza C virus.

20. An antibody that specifically binds to an epitope from a polypeptide selected from the polypeptides of SEQ ID NO:'s 4; 6; 8; 10; 12; 14; and 16-20.

21. A kit for detecting a Bovine Influenza C virus, the kit comprising the antibody of claim 19 and means for detecting the antibody.

22. A kit for detecting a Bovine Influenza C virus, the kit comprising an antibody of claim 20 and means for detecting the antibody.

23. (canceled)


SEQUENCE LISTING (20 sequences)

SEQ ID NO: 1 - DNA, 448 bp, Artificial Sequence
  cDNA sequence fragment of Bovine Influenca C Virus
tttggacctggagtggacaattcttctgtaataagggatgtttacaaagcaaaatttatgaaaaaagagagatggagagagactctgtggggaccaatgaactttgaactggtgggtaaacaaagaagagtggttgaaactcaaccagtggaaataaaactaaaccaaaaagaaataaaagaactaacgatgtgggttctttttgaggatgaagcaaaccttgcaagcaaattcatacaggaaaatttctcacttgtcctgtcattaagagaactttacaagggaaaggcagtaaataaagatgttgcagcttttatgattgcacaccaattttctcccgagaagagattcttacccacttttggccccattagaccagaaaggatggagctactccactgtttagggggtgacttctgaaacatcggcgaccccataacttcagcaa

SEQ ID NO: 2 - protein, 139 aa, Artificial Sequence
  amino acid sequence of RT-PCR fragment of Bovine Influenza C virus
FGPGVDNSSVIRDVYKAKFMKKERWRETLWGPMNFELVGKQRRVVETQPVEIKLNQKEIKELTMWVLFEDEANLASKFIQENFSLVLSLRELYKGKAVNKDVAAFMIAHQFSPEKRFLPTFGPIRPERMELLHCLGGDF

SEQ ID NO: 3 - DNA, 2330 bp, Artificial Sequence
  cDNA of PB2 of Bovine Influenza C virus
ttaacgctcgcaaaagagtatgcaaatctcacaaaggataagaaatcatgcaaattgctgtcgcaagggaccgtgtcaagctacaccacttttaagaaatggacaacatcgagaaaagagaaaaacccatcccttcgaatgagatgggcaatgggaagcaagttcccaatcatggcaaacagagaaattttggaagaagctgggataccagaacaatgggaagggatagacctttggtcaaaaaaggatgatgtttcaaagttggggatggtgttagcgtctcccgcagccataacttactggaatttttgtggacctggagtggacaattcttctgtaataagggatgtttacaaagcaaaatttatgaaaaaagagagatggagagagactctgtggggaccaatgaactttgaactggtgggtaaacaaagaagagtggttgaaactcaaccagtggaaataaaactaaaccaaaaagaaataaaagaactaacgatgtgggttctttttgaggatgaagcaaaccttgcaagcaaattcatacaggaaaatttctcacttgtcctgtcattaagagaactttacaagggaaaggyagtaaataaagatgttgcagcttttatgattgcacaccaattttctcccgagaagagattcttacccacttttggccccattagaccagaaaggatggagctactccactgtttagggggtgacttctggaagatagaggcagtaactgcagggagtctgaatgaagaacaaaagaagagagatgttagagcagttgccagaaaaatttgccttagagcaagtgtggacttatttactccagcagagaagataagggactatatatcaaatgtgacaatgagatttggaacagtagaaagaacgttcgaagacgtaataagaaacagtgatgatatatctgcggaagtgaccttatgcaaagcggcacttggatgcgaattaggcaaaagcatgagctttgggaatctaaatctgaggaaagtcagtggagaagcagaaacaatagaaaaaacagtatattggggattaaagcccataaaatacaaatgctggagaggagaggaaacattttattgcgaactgaggaaagtaacttgtatgtttagaaggtctgaaggcctagattgggctaacattggacctggttcacctgaagaaagaagagagctcttggcaatggtgatgatattctgcagagatgggagattctttgaatctgcaccagttaatattgatgaatcattctttagaacaagactgaataaagaaataccttatcaatatgtgctgctaaaatgggtaaggcaatcgagagacaacttggatgccttgttgagtacaagaggactgatacctgctcatattggacaattcggaaaaggaatgggaatagatggaagtagctcatcttctatggtttacaagggagtcatgttgtcgaaaacaccgatagacatagtggagagcaaagagaagcacaggctgtttttaaatgacaatatagaagcgataacagagagaggagcaatggttgcatccataatggacctatcagaggataatagagaaacatttaacgatgtgacttttaaccatgtcgacttagctgttctcaaagatgaaaaaactgcaataataaagatctatcgatcactggtggaaagaataaacactgatgatgatggcctacctgctttgataatgggtaaaagatatttagagttgtatcaattagatgaagtgaaagacgcggtcgggctaataccaaaacggatgctgggggcgtattcctaccaggcaagacagctcatacaatcgcagatcaaaaatgacagttatagccttcctgaaataataaagttgctgcccttctgttacagccctccaaagaaaatgttatttgatgggactttccatttcaaaaatcaaatgtatgttaggcctgggataaacacaaaccttttcagttttagtaagaccgacaaaagcaagatatatgtgaacggaagcgcagtgaaaataaagcttgtgctcggagacgatgaaatggacaccagtcttgcctttgttgaaggatttcaagtttgtgaatatgatccaagagcacctttgataccaagaagagatttgagactgattgggttcggaaagaaagttagagtttttgttggtcagggacaggagaaaaccctggtgaggacgagctccaaaagagccgcctcccatgatgtaagcaaaaacattcgtagaatgcgtctggaagtttgaagcacattgaaaaaatcctcttg

SEQ ID NO: 4 - protein, 744 aa, Artificial Sequence
  PB2 polymerase protein, Bovine Influenza C virus
VSSYTTFKKWTTSRKEKNPSLRMRWAMGSKFPIMANREILEEAGIPEQWEGIDLWSKKDDVSKLGMVLASPAAITYWNFCGPGVDNSSVIRDVYKAKFMKKERWRETLWGPMNFELVGKQRRVVETQPVEIKLNQKEIKELTMWVLFEDEANLASKFIQENFSLVLSLRELYKGKXVNKDVAAFMIAHQFSPEKRFLPTFGPIRPERMELLHCLGGDFWKIEAVTAGSLNEEQKKRDVRAVARKICLRASVDLFTPAEKIRDYISNVTMRFGTVERTFEDVIRNSDDISAEVTLCKAALGCELGKSMSFGNLNLRKVSGEAETIEKTVYWGLKPIKYKCWRGEETFYCELRKVTCMFRRSEGLDWANIGPGSPEERRELLAMVMIFCRDGRFFESAPVNIDESFFRTRLNKEIPYQYVLLKWVRQSRDNLDALLSTRGLIPAHIGQFGKGMGIDGSSSSSMVYKGVMLSKTPIDIVESKEKHRLFLNDNIEAITERGAMVASIMDLSEDNRETFNDVTFNHVDLAVLKDEKTAIIKIYRSLVERINTDDDGLPALIMGKRYLELYQLDEVKDAVGLIPKRMLGAYSYQARQLIQSQIKNDSYSLPEIIKLLPFCYSPPKKMLFDGTFHFKNQMYVRPGINTNLFSFSKTDKSKIYVNGSAVKIKLVLGDDEMDTSLAFVEGFQVCEYDPRAPLIPRRDLRLIGFGKKVRVFVGQGQEKTLVRTSSKRAASHDVSKNIRRMRLEV

SEQ ID NO: 5 - DNA, 2284 bp, Artificial Sequence
  PB1 cDNA of Bovine Influenza C virus
cccatatctactcatgttaaacaatgatataacgtcaatgatatcattgacatacccatacactggggcacctcctatgtcacatggaacatcgaccaaatactcaatggagactgtgtcaagaacttactcctacagtagaactaaaaaagaggtaccttcgggaatattcccaatagagagaaggaaattctgcaacacaatggaagacaaagaaaacctggaaaaaccgaatggaaatgttgatataaatttcatgttatcgctggcagaaatgctggaagaaaagatggggaaagggttcttcaaattctgtgccaatgaagctgaagctgaaattttgaagatgcacttcagcaagctcactgaaggaaggcaaacttatgactggacaagtgaaagaaatatgccagcagccaccgctcttcagctaacagtagacgctatacaagaaacacagggaacattcaaaggaactaccatggttgaatattgcaacaagatattagaaatgatggattggccagaagtgaaattcaaaaaggtcagaatgattgttcagaggcattgggacccgaaaaccaaaaaagaaataaaaatgaagtctccaacattgatgataacaaagattggaagagaagaattcataaagaggatatgcacgataaacaccatggccaaagacggagaaagaggaaaatacaaaagaagagctatagccactcccgggatgggaatcaggccattctcaaaaattgtggaaactttagcacaaaagatttgtgagagactagcagagagcggtttgcctgttgggggaaatgagaagaaagccaagctaaaaactacggtctcttcaacaaactcaaaattacaagaagggcagttcatggtaaacataacaggggacaacagcaagtggaatgaatgtcagcaaccagaagcttatcttgcaatgttggcatatattactaaagacagcagcaacttaatgaaagatctctgctcagtagcaccaacattgttctgcaataagtacgtgaaaatgggacaaggtttccgagcaaaaaacaaaagaaaaaccaaagaaatagtgatacccgcaaaaaggatgaaagaaaggaaagaattgatgaacgcggaatggagggacctatttgaaacaatagaaccttacatggatggagagtgctgcttcttggggggaggaatgctgatgggaatgtttaacatgttgtcgactgtttttggagtcatgacattaaattacagggaggaagcattggccagaaggaactgttactggactgggctacaaagttcagatgattttgtgctcttttgcatctctaggacttggccagagatggagatgactattctaaaattcatcgctgtttgcaagttgatgggaataaacatgtctttggaaaaatcctacgggtgcttgcctgaactctttgaattcacaagcatgttcttttccggggattttgtctcaaacatagccttggagttacctgctttcacaacagctggaatgaatgaaggaaccgacttcacagctgcgatgtctgtcataagaacaaacatgatcaataatggactttctcctgggactgctttaatggccctgcgaatttgtctgcaggaatttagagcaacatacagagtacacccttatgattctggagtgaagaatcatcgaatgaaaatcataaggaaattcattgaaactattgaaaacaaagatggattgctgatatcagatggcgggaaattaatgaacaatatctcaagtttgcacatccctgaagaaatattgaaagaggatttgatggatccctcctacaggaacagagttttcaatcctaggaacccctttacacagtttgagaagacagttgacatctttaaggcaagtgggcctataagggtagaggagaacgaggcagttgtatcaacgcattcctttagaacaaggagcaataggacattgctaaatacagacatgagagcaatggctctcgaagagaaaagataccaagttgtttgcaacatgtaccgatcgatcttcgaaagtgcagacgttaacaccccaataggatcaatgtcgatgggagaggcaattgaagccaaaatccttgaccgggccagaacccagtttgaaaatggaatcatagggggagaagaatattctgaaatcaaaagactaatcgaggacgccaagcgtcaacgactgtctgtttaagctttgcattgtttaataacagaaaaatcctct

SEQ ID NO: 6 - protein, 745 aa, Artificial Sequence
  PB1 polymerase protein of Bovine Influenza C virus
MLNNDITSMISLTYPYTGAPPMSHGTSTKYSMETVSRTYSYSRTKKEVPSGIFPIERRKFCNTMEDKENLEKPNGNVDINFMLSLAEMLEEKMGKGFFKFCANEAEAEILKMHFSKLTEGRQTYDWTSERNMPAATALQLTVDAIQETQGTFKGTTMVEYCNKILEMMDWPEVKFKKVRMIVQRHWDPKTKKEIKMKSPTLMITKIGREEFIKRICTINTMAKDGERGKYKRRAIATPGMGIRPFSKIVETLAQKICERLAESGLPVGGNEKKAKLKTTVSSTNSKLQEGQFMVNITGDNSKWNECQQPEAYLAMLAYITKDSSNLMKDLCSVAPTLFCNKYVKMGQGFRAKNKRKTKEIVIPAKRMKERKELMNAEWRDLFETIEPYMDGECCFLGGGMLMGMFNMLSTVFGVMTLNYREEALARRNCYWTGLQSSDDFVLFCISRTWPEMEMTILKFIAVCKLMGINMSLEKSYGCLPELFEFTSMFFSGDFVSNIALELPAFTTAGMNEGTDFTAAMSVIRTNMINNGLSPGTALMALRICLQEFRATYRVHPYDSGVKNHRMKIIRKFIETIENKDGLLISDGGKLMNNISSLHIPEEILKEDLMDPSYRNRVFNPRNPFTQFEKTVDIFKASGPIRVEENEAVVSTHSFRTRSNRTLLNTDMRAMALEEKRYQVVCNMYRSIFESADVNTPIGSMSMGEAIEAKILDRARTQFENGIIGGEEYSEIKRLIEDAKRQRLSV

SEQ ID NO: 7 - DNA, 2179 bp, Artificial Sequence
  PA cDNA of Bovine Influenza C virus
caggagatttagaaatgtctagtgtaatcagagaaatcgcaaagcgattcttggaacaagcaacgataaacatcgctgaagaagtggtcagagaatatggagaccatgaaagaacaatgatatctgttggagttcatttccaagcttgctgccttataagtgatgaatataccctcgaggatgagacaaccccaagatacgttcttttggagggattgagaagacaagaggctataagcaagcagaataacatttgctccactttgggattggaacccttgagaaatctagcagatatttttgatcgaaaaacaagaagattccttgaagtaggaattacaaagagagaatccgatgagtattaccaggaaaagttcaacaaaataggaaatgacatggatatacatgttttcacatatgaaggcaaatatttcagcaacaatcccaatgggttggaagacatccaaaagacaagaatttttacattcctatcttttgtgtcagacgaattgagaaaagagaacatgttcacagaaatgtatgttacagaagaaggggcacctgagcttgaaatgtacaagtcaaagcttttcattgcaatgagagacgagagcgtgcctttgccttacataaactatgagcaccttaggacaagatgtgaaacattcaaaagaaatcaggctgaatgcgaagcaaaggtagcggatgtggcttcacggctaaaaatcaaactggaacatctagaagaaaataaactgcgaccgctagagataccgaaggagaaagaggctccctatacgcacaaatttatgatgaaagatgcttggttctttgcaaaacctcatgattcggagagagcacaaccgcaacagatattgtatgatttctttgaagcagcaaacatggggttcatgacgacatccccaaaaccgatattcggaaagcaaggactgatgtatcactccctctgggggcagataaaaagggcaataaaggacaagagaaatgagttggagccttcagaacagagggacttcctttgtggaattggaagagcctccaagaaaatacaggaggacaaatggcaagaatccagagaggaagagtttaaacaagaagagactaaaggggcagctaagagggggttcccaacatggtttaatgaagaatggctttgggcaatgagggactcaggggatggggacaacaaaataggggattggatacccatggcagaaatgcctccctgcaagaatgagatggaagattatgcaaaaaagatgtgtgaagaattagaatccaaaatacagggaacaaattgtgctagggaaatgtccaagttgatacatacaattgggagcttacatacagaatgtaggaactttcccggaaaggtcaagatagtgcctatatactgcagagggacactgagaggggaatcaactgactgtttgtttggaatagcaataaaagggaaatcccatttaaacaaagatgatggaatgtatactgttgtaacttttgagttttccactgaagaaccaaatccaagcaaacatgaaaaatatacggtcttcgaagctggaacagtgcctgtggaagccgtggtgttaactcccaaaagggaaagagttctcaaagagaagaaattgtttctttattgcagaactactggaatgagcaagttaaagaacgattggttttctaaatgcaggagatgtcttataccaacaatggagactgtagagcagatagtgctgaaagaatgcgctctgaaagaagaaaacagagtttcagagatgttggagaataagagagcttggattgcccatgagaacggagagaatcttacaagattggtatcaacaaagctcaaagacttgtgtagaatgctaattgtgacacaattttattactgtatatataacgacaatcagttggaaggattctgtaacgagcaaaagaaattccttatgtttcttcaagcagataaggactcaaaatctgcatttacttttaatcagaaagggttatatgaaaaaattgaagagtgtattgtcagcaatccattatgtattttcctagctgataggctaaacaaattatttcttgtagccaagtccaatggagctaagtactttgaatgaccaaaggccttgtaatgttaaaaatctccttg

SEQ ID NO: 8 - protein, 710 aa, Artificial Sequence
  PA protein of Bovine Influenza C virus
MSSVIREIAKRFLEQATINIAEEVVREYGDHERTMISVGVHFQACCLISDEYTLEDETTPRYVLLEGLRRQEAISKQNNICSTLGLEPLRNLADIFDRKTRRFLEVGITKRESDEYYQEKFNKIGNDMDIHVFTYEGKYFSNNPNGLEDIQKTRIFTFLSFVSDELRKENMFTEMYVTEEGAPELEMYKSKLFIAMRDESVPLPYINYEHLRTRCETFKRNQAECEAKVADVASRLKIKLEHLEENKLRPLEIPKEKEAPYTHKFMMKDAWFFAKPHDSERAQPQQILYDFFEAANMGFMTTSPKPIFGKQGLMYHSLWGQIKRAIKDKRNELEPSEQRDFLCGIGRASKKIQEDKWQESREEEFKQEETKGAAKRGFPTWFNEEWLWAMRDSGDGDNKIGDWIPMAEMPPCKNEMEDYAKKMCEELESKIQGTNCAREMSKLIHTIGSLHTECRNFPGKVKIVPIYCRGTLRGESTDCLFGIAIKGKSHLNKDDGMYTVVTFEFSTEEPNPSKHEKYTVFEAGTVPVEAVVLTPKRERVLKEKKLFLYCRTTGMSKLKNDWFSKCRRCLIPTMETVEQIVLKECALKEENRVSEMLENKRAWIAHENGENLTRLVSTKLKDLCRMLIVTQFYYCIYNDNQLEGFCNEQKKFLMFLQADKDSKSAFTFNQKGLYEKIEECIVSNPLCIFLADRLNKLFLVAKSNGAKYFE

SEQ ID NO: 9 - DNA, 2095 bp, Artificial Sequence
  HE cDNA of Bovine Influenza C virus
atcaaatgtgatataagtccataatccttcatctgtgattctcaattcaaaaagccctaaacagcaggagattttcaaagatgtttttgcttctagcaacaattacagcaataactgcttgccaagcagaaagagaactgatatgtatagtgcaaagagtgaatgaaagcttctctcttcactctggatttggaggaaatgtttacagcatgaagactgagccaattgctggattcacaaatgtgaccaaaggtgctagtgtcatcaaccaaaaagactgggttggattcggagattcaaggacagacttgactaatgctcagtttccagcgtcttcagatgtcccattggccgtggcamagaagtttaggtcattgtcaggggcttcgttaatgttgtcagcttttgggcctcctggcaaggttgattacctttatcaaggatgcgggaaagaaaaagtattttatgaaggggtaaactggtcccctgaggcaggaattgattgctttgggtcaaactggactcagacaaagaaggacttctattcgaggatatatgaagctgctagaggcagcacatgcatgacccttgtaaattctctagacaccaagatatcatcaaaaacagccacagctggaaccacatcttcttgttcttcaagctggatgaaaagcccgttgtggtatgcagagtcttctgttaatcctggagctagacctcaagcttgtgggactgagcaatcagcaacttttactttgccgacaagtttcggaatttacaaatgcaacaagcatgtagtgcaactttgttactttgtgtatgaaaacaaaacagcatttaacactcttggctgtggagattattaccaaaattactatgacgggaatggaaacctggtagggggagtggataacagagtggcagcatacagaggaatagcaggctctggagttaaaattgaatgtccttcaaaaatcttgaaccctgggacttacagcattagatcaacaccaaaattccttctggtaccaaaaaggtcatattgcttcgacactgatggagggtaccctatacaagtggttcaatctgagtggtcagcttcacgaagatcagataawgccactgaagaagcatgcctacaaacagaaggatgtattttcatcaaaaagacaaccccttatgtaggagaagcagatgacaaccatggggacattgagatgaggcaactcttgagtgggcttggcaacaacgacacagtgtgcgtttcccaaagtggatacacaaaaggagagaccccttttgtaaaggattatttgagtcctcccaagtatggcagatgtcagttgaaaactgacagtggaagaatcccaactctaccttctgggttgataataccgcaagcagggactgattctttaatgagaacyttgacaccagcaacaaggatcttcggaatagatgacttaatcttcgggcttttattcatagggtttgtcgcaggaggagttgcaggaggttacttctggggaagatcaagtggagggggtggtggtgcctcggtgagcagtacgcaggctggatttgacaaaatcggaaaagatatacagcagcttcggaatgacacaaatgcagcaattgaaggcttcaacgggagaattgcccatgatgagcaagccattaagaatttggcaaaagaaatcgaagatgcaagggcagaagctttggtaggggaacttggtataataagatccctcatagttgccaacataagcatgaatctaaaagaatctttatatgaactcgcaaaccaaataacaaagagaggaggaggaattgcacaagaagcaggcccagggtgttggtatgttgactccgaaaactgtgatgcaagctgcaaggagtacattttcaacttcaatggcagtgccactgtccccacattgaggccagttgacaccaatgttgtaataacatcggatccttattacttgggctcgaccatagctctctgtcttttgggtctaatggcgattgctgcttttgttggtgtgagttggatctgttgcaagaaatagaatcttagaaaaaatctcct

SEQ ID NO: 10 - protein, 664 aa, Artificial Sequence
  hemagglutinin-esterase-fusion protein of Bovine Influenza C virus
MFLLLATITAITACQAERELICIVQRVNESFSLHSGFGGNVYSMKTEPIAGFTNVTKGASVINQKDWVGFGDSRTDLTNAQFPASSDVPLAVAXKFRSLSGASLMLSAFGPPGKVDYLYQGCGKEKVFYEGVNWSPEAGIDCFGSNWTQTKKDFYSRIYEAARGSTCMTLVNSLDTKISSKTATAGTTSSCSSSWMKSPLWYAESSVNPGARPQACGTEQSATFTLPTSFGIYKCNKHVVQLCYFVYENKTAFNTLGCGDYYQNYYDGNGNLVGGVDNRVAAYRGIAGSGVKIECPSKILNPGTYSIRSTPKFLLVPKRSYCFDTDGGYPIQVVQSEWSASRRSDXATEEACLQTEGCIFIKKTTPYVGEADDNHGDIEMRQLLSGLGNNDTVCVSQSGYTKGETPFVKDYLSPPKYGRCQLKTDSGRIPTLPSGLIIPQAGTDSLMRTLTPATRIFGIDDLIFGLLFIGFVAGGVAGGYFWGRSSGGGGGASVSSTQAGFDKIGKDIQQLRNDTNAAIEGFNGRIAHDEQAIKNLAKEIEDARAEALVGELGIIRSLIVANISMNLKESLYELANQITKRGGGIAQEAGPGCWYVDSENCDASCKEYIFNFNGSATVPTLRPVDTNVVITSDPYYLGSTIALCLLGLMAIAAFVGVSWICCKK

SEQ ID NO: 11 - DNA, 1753 bp, Artificial Sequence
  N cDNA of Bovine Influenza C virus
attattaagcaatatggactcaacaaaagcccaaacgcctgaagagcaaagagcaaagaatgccaaaaccatccttgagaacatacagatatatgaaaggatgtgtgatctctttggggtgtcagaagatgacaaactgataattgagaacagtatttcaattgagagaatgataagagttgtaacagataagaaatatcaagacaagaaactaaagaatgctggaagtgatcttgaaaagattgcaaatgcagggaaagttttctgccgattggtggagtcaacagctgggaaatgtagtgcaagattgggaatggcactgaaacccaacgttgaggcagtcctgaccgatgtactcgggaatgaactggatagggctgctgtgcttgggaaaaggatggggtttacagcaatgttcaaatcaaacctggaagaggttttgtaccaaagaggaaagaatcagcttaaaaagaggaatgctgcagaaactttcactctttcacaaggtgcttcgctagaggcaagatttaggcccataatggaaaaacacctaggtgttgggactgttgtggcgtcaataaagaatatcctggcaagcaaaaagaacgggaactacaggaacaagatggtgaggaaacctggaggaagcagagaaagctggtcaccattggagagagaaatatcctttctgaacaagaagttgtttcctggaccaatgaggcagctctgcaagaaattcgaatacttgaacgaccaagagaagcaactggccttaaacctcatgctggatgcaagtctcatcctgaagccacaagtgactcataaaatgataatgccttggtcaatgtggctggctgtgaagaaatatgcagaaatgaacaagggatcacccagtcttgaagacctcgcagcctattctggagtaagagcctttatggccttcaatacagcttgctacatgagtaaattcaccattgggaagggaattgtgggagatgcagagatcatggaaaatggaaacgacaagatgcaaactcttgcaatggcttgttttggactggcgtatgaagacaccgggattgttgcagcaatgatctcccaacccatgaagaaaagatatcagttgagagtggggaacttcaaccctccagaagaaggaacaataaaaggaacaagcgccggctatttccacaagtgggctgaatttggaaataggctgcctttcaacagttttggaactggtgaatccaaacagataagcaactcaggagtgtttgcagtgcagaggcccagcactactaacattcaaagactggcagagctaatggctaggaatactggagaaaccagcgacaactttactcagttggttcagaaaataagagaacaagtggggacctttgctgatcaaaaagcaaatcttcgagagttcaccggaggatatatttatgatattactgacgtaacgaagagcaaccccaagatacctcagttgggtggggactctttcttctttgagttcaccggaagcgacgttccaagaactggagccaaaagaagagtgggaggagctgatgatgtgacccctggaacttcccagcccaagaaaagaggaaggcaaggtgccggagcagaatcaagtatggacattgaaacagttggtgaagattaactcatctttggatggttctgttgcagtccccagtgggtgttaaaggtgttggtttgtcttatttaacaaaaaatctccttg

SEQ ID NO: 12 - protein, 552 aa, Artificial Sequence
  Nucleocapsid protein of Bovine Influenza C virus
MDSTKAQTPEEQRAKNAKTILENIQIYERMCDLFGVSEDDKLIIENSISIERMIRVVTDKKYQDKKLKNAGSDLEKIANAGKVFCRLVESTAGKCSARLGMALKPNVEAVLTDVLGNELDRAAVLGKRMGFTAMFKSNLEEVLYQRGKNQLKKRNAAETFTLSQGASLEARFRPIMEKHLGVGTVVASIKNILASKKNGNYRNKMVRKPGGSRESWSPLEREISFLNKKLFPGPMRQLCKKFEYLNDQEKQLALNLMLDASLILKPQVTHKMIMPWSMWLAVKKYAEMNKGSPSLEDLAAYSGVRAFMAFNTACYMSKFTIGKGIVGDAEIMENGNDKMQTLAMACFGLAYEDTGIVAAMISQPMKKRYQLRVGNFNPPEEGTIKGTSAGYFHKWAEFGNRLPFNSFGTGESKQISNSGVFAVQRPSTTNIQRLAELMARNTGETSDNFTQLVQKIREQVGTFADQKANLREFTGGYIYDITDVTKSNPKIPQLGGDSFFFEFTGSDVPRTGAKRRVGGADDVTPGTSQPKKRGRQGAGAESSMDIETVGED

SEQ ID NO: 13 - DNA, 1203 bp, Artificial Sequence
  Matrix cDNA of Bovine Influenza C virus
cagaggatatttttgacgcaatggcacaagaacaactacttgctgaacttgagggatacctcagaggaattaacccaatgaccaggcaaacaattttgaggtctgcaagaggtggaatggatagcgctagggatgcagcaagagcagcgaagaagggagaaatgcaactaacaagtggagagagtatagtggtgcacatatgcctgagggccatgtaccctggaataaagccatggtcagaagccaaaaaagatcttgataaagctacagaaggactgagtggaagagacagcaaaagcataagaaaggctttgaggaaggcgggagacctgacagggataaaggagatgatgatgatgtatgaaatgagggaggacaaaaaagcagaaatggtggagcaaatctacgacgatccagaggattttacagaagatgtaaggcttgggaccgttgctgcctggcttcaatgcaaaaacaaaaggagcgagaaatatcatcacaagatgtcaatgtctggaagcactgcacttgccttgggagacgcccaaaaggccggaatggccatagaaaatatggccagtgttgtaccaatgaaaaaagaggcccaggcactgcacaaagacgcagaagtcttaattgaactggcaagaatagcatatgggtcaagagcaatggaagggcacctgcaaaatgcaatggacggaattggaagcaaagtcagtggaatggctaatcttgctctaaaaaggtcagttctcactttgttaatgttggtaatttgtgggatatccacctgtgtaaatgctgaaactgtggaagaattttgtagaaagaaactaaatcagacggaagaaaaggtttatgtccattttttcaatgaggatgatagtcgggcaatgactctagctgctttgatacttggatgctttagtatgctttacattttaataaaggcaatactgatgtttttgttgacaatcataaatggaagaccaaatggaagttgggatgacttgaaacatgttgtaaaatgtttttcagagactggaagtgagaacttcgccagggatataatggtcctggagtccaggcgagatggggaggagacaagctccctggaggagggactgggccctccattgagtggattcaatgaaaatggtgtattcatggaaacattataatcgcgaaaaaatcctcttg

SEQ ID NO: 14 - protein, 387 aa, Artificial Sequence
  Matrix protein of Bovine Influenza C virus
MAQEQLLAELEGYLRGINPMTRQTILRSARGGMDSARDAARAAKKGEMQLTSGESIVVHICLRAMYPGIKPWSEAKKDLDKATEGLSGRDSKSIRKALRKAGDLTGIKEMMMMYEMREDKKAEMVEQIYDDPEDFTEDVRLGTVAAWLQCKNKRSEKYHHKMSMSGSTALALGDAQKAGMAIENMASVVPMKKEAQALHKDAEVLIELARIAYGSRAMEGHLQNAMDGIGSKVSGMANLALKRSVLTLLMLVICGISTCVNAETVEEFCRKKLNQTEEKVYVHFFNEDDSRAMTLAALILGCFSMLYILIKAILMFLLTIINGRPNGSWDDLKHVVKCFSETGSENFARDIMVLESRRDGEETSSLEEGLGPPLSGFNENGVFMETL

SEQ ID NO: 15 - DNA, 852 bp, Artificial Sequence
  NS cDNA of Bovine Influenza C virus
caggggtgtacaatttcaatatgtctgaaaataagtcagtgaacacaacaaatatcagagcagcaatctccgaattggcattaggcgcagccagctggatggattcctctggattaatgactttcgagaaaatgagaaagtctgctgagaattcactgagagtcgaacaggtttatgaaccgagaacttgggaagatgcagtggccgaaggtagagaaattctaggattcactactattgctgccttaagaaaaccagaggagactcatgctgttgaattgggaaagaacattatctatcccttaggaggaaatcctttctatctgagcccatgtaccattgacactctgtatgagccaaagctcataagacaaggagaagtcttgggagtaaaatatcggaactgcaattgctttgtaaaaactgctgaactattagtgaccgacatgggagaaatcattgtgctcttttgcagaaacactgagaaaccagcttactgccttaagaatttccgtagaggagatgacccagagaagtcagtacgaaagatactcagaatttggagaagtggacttgttgttgccgttgatgcggaatctagagatgagatcagacgatacaaatctggatgtgaaacagatcccttctggagaagagaaggcgcaactactggagaggttcaggagttgcttggtgtcattgataaggttgaaatccaagctgggagtagcgatggtgaactctttgactaaccaggatatgagggccgctttggatgaaatcaagtcagtttcgagaacaatttcaatgttaaaagaatgtattcgttctttagtatgaaaaaaccccttg

SEQ ID NO: 16 - protein, 248 aa, Artificial Sequence
  non-structural protein of Bovine Influenza C virus
VYNFNMSENKSVNTTNIRAAISELALGAASWMDSSGLMTFEKMRKSAENSLRVEQVYEPRTWEDAVAEGREILGFTTIAALRKPEETHAVELGKNIIYPLGGNPFYLSPCTIDTLYEPKLIRQGEVLGVKYRNCNCFVKTAELLVTDMGEIIVLFCRNTEKPAYCLKNFRRGDDPEKSVRKILRIWRSGLVVAVDAESRDEIRRYKSGCETDPFWRREGATTGEVQELLGVIDKVEIQAGSSDGELFD

SEQ ID NO: 17 - protein, 36 aa, Artificial Sequence
  a fragment of M protein of Bovine Influenza C virus
EMVEQIYDDPEDFTEDVRLGTVAAWLQCKNKRSEKY

SEQ ID NO: 18 - protein, 79 aa, Artificial Sequence
  a fragment of hemagglutinin-esterase-fusion protein of Bovine Influenza C virus
GASVINQKDWVGFGDSRTDLTNAQFPASSDVPLAVAXKFRSLSGASLMLSAFGPPGKVDYLYQGCGKEKVFYEGVNWSP

SEQ ID NO: 19 - protein, 60 aa, Artificial Sequence
  a fragment of hemagglutinin-esterase-fusion protein of Bovine Influenza C virus
EIEDARAEALVGELGIIRSLIVANISMNLKESLYELANQITKRGGGIAQEAGPGCWYVDS

SEQ ID NO: 20 - protein, 44 aa, Artificial Sequence
  a fragment of hemagglutinin-esterase-fusion protein of Bovine Influenza C virus
AXKFRSLSGASLMLSAFGPPGKVDYLYQGCGKEKVFYEGVNWSP